(19) United States
(12) Patent Application Publication
(10) Pub. No.: US 2020/0024662 A1
(43) Pub. Date: Jan. 23, 2020

(54) METHODS FOR TREATING OPIOID ADDICTION

(71) Applicant: ASSUREX HEALTH, INC., Mason, OH (US)

(72) Inventors: Bryan DECHAIRO, Mason, OH (US); David LEWIS, Mason, OH (US); Alexa GILBERT, Mason, OH (US); James LI, Mason, OH (US); Balmiki RAY, Mason, OH (US); Rebecca LAW, Mason, OH (US)

(73) Assignee: ASSUREX HEALTH, INC., Mason, OH (US)

(21) Appl. No.: 16/463,347

(22) PCT Filed: Nov. 22, 2017
(86) PCT No.: PCT/US2017/063032

§ 371 Date: May 22, 2019

Related U.S. Application Data
(60) Provisional application No. 62/425,894, filed on Nov. 23, 2016.

(51) Int. Cl. C12Q 1/6876; A61P 25/36; A61K 31/485; A61K 31/137; G16B 40/00; G16B 5/00

(52) U.S. Cl. CPC C12Q 1/6876; A61P 25/36; A61K 31/485; C12Q 2600/106; G16B 40/00; G16B 5/00; A61K 31/137

(57) ABSTRACT

The invention provides methods and compositions for treating opioid addiction by identifying patients at high risk of failing opioid agonist replacement therapy before therapy has begun. Related compositions, in the form of kits, systems, and computer-readable media are also provided.

### RELATED APPLICATIONS

The present application is filed under 35 U.S.C. § 371 as the U.S. national phase of International Patent Application No. PCT/US2017/063032, filed Nov. 22, 2017, which designated the United States and claims priority to U.S. Provisional Application No. 62/425,894, filed Nov. 23, 2016, the entire contents of each of which are hereby incorporated by reference in their entirety.

### FIELD OF INVENTION

The present invention relates to the fields of medicine and psychiatry. In particular, the invention relates to methods and compositions for treating opioid addiction using opioid agonist replacement therapy.

### BACKGROUND OF THE INVENTION

Opioid misuse and addiction and is a serious and growing public health problem. The current standard treatment for opioid addiction is opioid agonist replacement therapy, either using methodone alone or a combination of buprenorphine and naloxone (this combination is also commonly referred to by its tradename, “Suboxone™”). Typical treatment regimens require patients to receive the agonist replacement for a sustained period of time, e.g., 24 weeks. Although these treatments can be effective in reducing opioid dependence, there is a wide variation in effectiveness among patients. Many fail to complete the full 24 weeks of treatment because they are unable to remain abstinent dropout before completion of the program. Currently, there are no reliable predictors of an individual's likelihood of treatment retention, meaning the individual's ability to stay in treatment for the entire therapy period. Yet, treatment retention itself is a recognized predictor of favorable outcome. Accordingly, improving treatment retention is an important goal for improving outcomes. See e.g., Hsu et al. (2013). One way to improve treatment retention is to identify those patients who are at high risk of dropout before treatment begins, in order to target those patients for early interventions that may help them avoid dropout. Some factors tending to increase retention have been identified, such as dose and type of agonist, with higher doses generally correlating with increased retention and methadone therapy generally correlating with higher retention than buprenorphine. See id. But it is not possible using current methods to identify in advance which patients will require a higher dose, or a different drug, in order to improve their treatment retention. Indeed, treatment retention is a complex trait, most likely involving both genetic and environmental components, as well as gene-environment interactions, and there is currently no reliable predictor to identify patients who are at high risk of dropout before beginning therapy.

Crist et al., recognizing the role that genetic differences may play in the high variability in treatment outcome, looked at the relationship between genetic variants in OPRD1, the gene encoding the δ-opioid receptor, and the prevalence of opioid-positive urine tests (as a measure of abstinence, and indirectly treatment outcome in African-Americans or European-Americans undergoing treatment with either methadone or Suboxone™. Crist et al. Neuropsychopharmacology. 2013 September; 38(10): 2003-2010. Crist used data from an open-label clinical trial, ‘Starting Treatment with Agonist Replacement Therapy’ (START). START was a randomized, open-label, outpatient-based study assessing changes in liver enzymes related to treatment with methadone or Suboxone™ in order to assess the effects on liver function on treatment outcome. Saxon et al., Drug Alcohol Depend. 2013 Feb. 1; 128(1-2):71-6. The START trial also collected genetic material and other data on treatment response. The results of the Crist study suggested that the intronic SNP rs678849 predicted treatment outcome (abstinence) for both medications in African Americans.

Other studies have suggested a role for certain variants in the ABCB1, ANKK1, and DRD2 genes in optimal methadone dose (Crettol et al, Clin Pharmacol Ther. 2006 December; 80(6):668-81.; Hung et al, Pharmacogenomics. 2011 November; 12(11):1525-33.); and others have suggested that the ARRB2 and cytochrome P450 genes are associated with variability in response to methadone (Levran et al, Addict Biol. 2013 July; 18(4):709-16.; Oneda et al, Pharmacogenomics J. 2011 August; 11(4):258-66.).

None of the studies to date have been able to identify reliable genetic predictors of patients who are at high risk of dropout before beginning therapy. Accordingly, there is a need to identify those patients who are at high risk of dropping out of opioid agonist replacement therapy so that those patients can be targeted for early interventions to prevent dropout, increase treatment retention, and improve outcomes. Ideally, methods are needed which can reliably identify those patients at high risk of dropout before treatment begins, since dropout rates have been documented to be much higher during the initial 30 days of treatment, making it difficult to identify these at-risk patients in time to intervene and prevent their dropping out. See Hsu et al.

### SUMMARY OF THE INVENTION

The disclosure provides methods, including computer implemented or computer assisted methods, and compositions, including computer program products and computer systems, for treating patients for opioid addiction using opioid agonist replacement therapy. The methods and compositions described here enable the early identification of patients who are at high or intermediate risk of not completing opioid agonist replacement therapy. Identifying these patients, preferably before the beginning of therapy, means they can be targeted for early interventions to prevent dropout, increase treatment retention, and improve therapeutic outcomes.

In embodiments, the methods described here provide an improved treatment regimen for opioid addiction using opioid replacement therapy by including a genetic screen to identify patients at high, moderate, and low risk of not completing the therapy. Preferably the genetic screen is performed prior to the beginning of therapy.

In embodiments, the disclosure provides methods of treating opioid addiction in a human subject in need thereof, the method comprising determining the subject's genetic phenotype for one or more pharmacodynamic genes selected from ADRA2A, COMT, HTR2A, OPRM1, and SLC6A4, and optionally determining the subject's genetic phenotype for one or more cytochrome P450 (CYP)genes selected from the group consisting of CYP1A, CYP2B6, CYP2C9, CYP2C19, and CYP2D6; generating a composite genetic risk score for the subject which is the sum of the risks associated with each genetic phenotype; identifying the subject as having a high, intermediate, or low risk of non-completion of opioid agonist replacement therapy based on the subject's composite genetic risk score as follows: if the composite genetic risk score has a p-value of less than or equal to 0.05 and an odds ratio greater than or equal to 1.5, the subject is at high risk; if the composite genetic risk score has a p-value of less than or equal to 0.05 and an odds ratio between 1 and 1.5, the subject is at intermediate risk; and if none of the above conditions are met, the subject is at low risk; and administering an opioid agonist to the subject who is identified as being at high or intermediate risk of non-completion as follows: administer an initial dose of buprenorphine that is higher than starting dose determined according to the clinical guidelines; or administer methadone to the patient.

In embodiments, the disclosure provides methods of identifying a subject at risk of non-completion of opioid agonist replacement therapy, the method comprising determining the subject's genetic phenotype for one or more pharmacodynamic genes selected from ADRA2A, COMT, HTR2A, OPRM1, and SLC6A4, and optionally determining the subject's genetic phenotype for one or more cytochrome P450 (CYP)genes selected from the group consisting of CYP1A, CYP2B6, CYP2C9, CYP2C19, and CYP2D6; generating a composite genetic risk score for the subject which is the sum of the risks associated with each genetic phenotype; identifying the subject as having a high, intermediate, or low risk of non-completion of opioid agonist replacement therapy based on the subject's composite genetic risk score as follows: if the composite genetic risk score has a p-value of less than or equal to 0.05 and an odds ratio greater than or equal to 1.5, the subject is at high risk; if the composite genetic risk score has a p-value of less than or equal to 0.05 and an odds ratio between 1 and 1.5, the subject is at intermediate risk; and if none of the above conditions are met, the subject is at low risk.

In embodiments, the genetic phenotype of each of the one or more CYP genes is a combination phenotype based upon the number of functional alleles at the genetic locus. wherein the subject's genetic phenotype is determined for a panel of two genes selected from the group consisting of
COMT, CYP3A4;
COMT, HTR2A;
COMT, SLC6A4;
HTR2A, CYP3A4;
SLC6A4, HTR2A; and
SLC6A4, CYP3A4.

In embodiments, the subject's genetic phenotype is determined for a panel of genes selected from the group consisting of
COMT, HTR2A, CYP3A4;
COMT, SLC6A4, CYP3A4;
COMT, SLC6A4, HTR2A; and
SLC6A4, HTR2A, CYP3A4.

In embodiments, the subject's genetic phenotype is determined for a panel of genes selected from the group consisting of
COMT, SLC6A4, HTR2A, CYP3A4;
COMT, SLC6A4, HTR2A, CYP3A4, CYP2B6;
COMT, SLC6A4, HTR2A, CYP3A4, CYP2C19;
COMT, SLC6A4, HTR2A, CYP3A4, CYP1A2;
COMT, SLC6A4, HTR2A, CYP3A4, CYP2C9; and
COMT, SLC6A4, HTR2A, CYP3A4, CYP2D6.

In embodiments, the subject's genetic phenotype is determined for a panel of genes selected from the group consisting of
COMT, SLC6A4, HTR2A, CYP3A4, CYP2B6, CYP2C19;
COMT, SLC6A4, HTR2A, CYP3A4, CYP2B6, CYP1A2;
COMT, SLC6A4, HTR2A, CYP3A4, CYP2B6, CYP2C9;
COMT, SLC6A4, HTR2A, CYP3A4, CYP2B6, CYP2D6;
COMT, SLC6A4, HTR2A, CYP3A4, CYP2C19, CYP1A2;
COMT, SLC6A4, HTR2A, CYP3A4, CYP2C19, CYP2C9; and
COMT, SLC6A4, HTR2A, CYP3A4, CYP2C19, CYP2D6.

In embodiments, the subject's genetic phenotype is determined for a panel of genes selected from the group consisting of
COMT, SLC6A4, HTR2A, CYP3A4, CYP2B6, CYP2C9, CYP2D6;
COMT, SLC6A4, HTR2A, CYP3A4, CYP2B6, CYP2C9, CYP2D6, CYP2C19, CYP1A2.

In embodiments, the genetic phenotype is determined by assaying for one or more of the following genetic variants in the one or more pharmacodynamic genes: ADRA2A (rs1800544), COMT (rs4680), HTR2A (rs6311), OPRM1 (rs1799971), SLC6A4 (5-HTTLPR).

In embodiments, the genetic phenotype is assigned based on the genotype at each genetic variant.

In embodiments, the one or more pharmacodynamic genes includes COMT and SLC6A4.

### BRIEF DESCRIPTION OF THE DRAWINGS

FIG. 1 shows a histogram of individuals in Sample 1 determined to be low risk (G), intermediate risk (Y), or high risk (R) by the baseline algorithm (Genesight Analgesic) in Example 2, sorted by actual completion status.

FIG. 2 shows a histogram of individuals in Sample 1 determined to be low risk (G), intermediate risk (Y), or high risk (R) by the PK Model in Example 2, sorted by actual completion status.

FIG. 3 shows a histogram of individuals in Sample 1 determined to be low risk (G), intermediate risk (Y), or high risk (R) by PD Model 1 in Example 2, sorted by actual completion status.

FIG. 4 shows a histogram of the percent of methadone treatment completers and non-completers in Sample 1 sorted by PD Model 1 risk score in Example 2.

FIG. 5 shows a histogram of individuals in Sample 1 determined to be low risk (G), intermediate risk (Y), or high risk (R) by PD Model 2 in Example 2, sorted by actual completion status.

FIG. 6 shows a histogram of individuals in Sample 1 determined to be low risk (G), intermediate risk (Y), or high risk (R) by PD Model 3 in Example 2, sorted by actual completion status.

FIG. 7 shows a histogram of individuals in Sample 1 determined to be low risk (G), intermediate risk (Y), or high risk (R) by PK/PD Model 1 in Example 2, sorted by actual completion status.

FIG. 8 shows a histogram of the percent of methadone treatment completers and non-completers in Sample 1 sorted by PK/PD Model 1 risk score in Example 2.

FIG. 9 shows a histogram of individuals in Sample 1 determined to be low risk (G), intermediate risk (Y), or high risk (R) by PK/PD Model 2 in Example 2, sorted by actual completion status.

FIG. 10 shows a histogram of individuals in Sample 1 determined to be low risk (G), intermediate risk (Y), or high risk (R) by PK/PD Model 3 in Example 2, sorted by actual completion status.

FIG. 11 shows a histogram of individuals in Sample 2 determined to be low risk (G), intermediate risk (Y), or high risk (R) by the baseline algorithm (Genesight Analgesic) in Example 2, sorted by actual completion status.

FIG. 12 shows a histogram of individuals in Sample 2 determined to be low risk (G), intermediate risk (Y), or high risk (R) by the PK Model in Example 2, sorted by actual completion status.

FIG. 13 shows a histogram of individuals in Sample 2 determined to be low risk (G), intermediate risk (Y), or high risk (R) by PD Model 1 in Example 2, sorted by actual completion status.

FIG. 14 shows a histogram of the percent of methadone treatment completers and non-completers in Sample 2 sorted by PD Model 1 risk score in Example 2.

FIG. 15 shows a histogram of individuals in Sample 2 determined to be low risk (G), intermediate risk (Y), or high risk (R) by PK/PD Model 1 in Example 2, sorted by actual completion status.

FIG. 16 shows a histogram of the percent of methadone treatment completers and non-completers in Sample 2 sorted by PK/PD Model 1 risk score in Example 2.

FIG. 17 shows a histogram of individuals in the Full Sample determined to be low risk (G), intermediate risk (Y), or high risk (R) by the baseline algorithm (Genesight Analgesic) in Example 2, sorted by actual completion status.

FIG. 18 shows a histogram of individuals in the Full Sample determined to be low risk (G), intermediate risk (Y), or high risk (R) by the PK Model in Example 2, sorted by actual completion status.

FIG. 19 shows a histogram of individuals in the Full Sample determined to be low risk (G), intermediate risk (Y), or high risk (R) by PD Model 1 in Example 2, sorted by actual completion status.

FIG. 20 shows a histogram of the percent of methadone treatment completers and non-completers in the Full Sample sorted by PD Model 1 risk score in Example 2.

FIG. 21 shows a histogram of individuals in the Full Sample determined to be low risk (G), intermediate risk (Y), or high risk (R) by PK/PD Model 1 in Example 2, sorted by actual completion status.

FIG. 22 shows a histogram of the percent of methadone treatment completers and non-completers in the Full Sample sorted by PK/PD Model 1 risk score in Example 2.

FIG. 23 shows a flow chart for assigning phenotypic scores to genotypes for use in PD Model 1. The phenotypic scores are then combined into a composite risk score (PD Model 1)

FIG. 24 shows a flow chart for assigning phenotypic scores to genotypes for use in PD Model 2. The phenotypic scores are then combined into a composite risk score (PD Model 2)

FIG. 25 shows a flow chart for assigning phenotypic scores to genotypes for use in PD Model 3. The phenotypic scores are then combined into a composite risk score (PD Model 3)

### DETAILED DESCRIPTION

The methods described here aim to identify, preferably before the beginning of therapy, those patients who are at high or intermediate risk of non-completion of opioid agonist replacement therapy for the treatment of opioid addiction. The terms “completer” and “non-completer” are used to refer to patients who complete, or who fail to complete, respectively, the full course of the opioid agonist replacement therapy. Typically, a full course of therapy will comprise 24 weeks of treatment at the full dose of the opioid agonist.

As described infra, the impact of various combinations of genetic markers on the ability of patients to complete a 24-week treatment regimen was assessed by generating a series of composite genetic risk scores based on combinations of genetic markers and determining whether any of the composite genetic risk scores had a significant association with non-completion. The individual genetic risk scores for each gene are summed to produce the composite score. For each gene, the risk score is based upon a “genetic phenotype” which is assigned using the patient's genotype or haplotype. The term “genetic phenotype” is meant to refer to the phenotype inferred or assigned based on genotype and/or haplotype alone, as distinct from the phenotype of the protein product of the gene which may be measured, for example, enzyme activity. The present methods do not require assays of protein function but are based on inferences from the genetic data. Thus, the methods may also provide a combinatorial genetic phenotype for the patient which is used to identify the patient's risk of non-completion. The combinatorial genetic phenotype may be a qualitative assessment based upon the patient's composite genetic risk score.

Throughout the present disclosure, the term ‘patient’ refers to a human subject who is need of treatment for addiction to opioids.

In embodiments of the methods described here, a patient in need of opioid replacement therapy is categorized according to the patient's risk of non-completion based on the patient's combinatorial genetic phenotype or composite genetic risk score. Accordingly, in embodiments, the methods comprise assaying a biological sample from the patient to determine the patient's genotype and/or haplotype at one or more genetic markers in one or more genes or genetic loci.

In embodiments, the methods described here optionally comprise assaying a biological sample from the patient for the presence of one or more non-genetic markers, such as the presence opiates, for example, in a urine sample from the patient.

In embodiments, the methods described here optionally comprise incorporating one or more non-genetic data attributes into the model for determining the patient's risk of non-completion. For example, the methods may incorporate data attributes such as ancestry (e.g., self-reported ancestry in one of a defined set of groups as described infra), prior failure of opioid replacement therapy, concomitant prescription drug use, concomitant illicit drug use, age, and gender. In this context, illicit drug use may refer to the abuse of illegal drugs or the misuse of prescription medications.

In embodiments, the disclosure also provides methods of reducing a patient's risk of non-completion of opioid agonist replacement therapy. In embodiments, the risk is reduced by identifying a patient as high or intermediate risk of non-completion before the start of therapy according to the methods described herein, and providing the at risk patient with one or more interventions and/or adjustments to the patient's therapy regimen aimed at increasing the chance of completion. For example, the patient may be prescribed an alternative agonist, e.g., methadone instead of buprenorphine, or an opioid antagonist, inverse agonist or alterative substance dependency medication, in order to increase the patient's likelihood of completing therapy. Other adjustments to the patient's therapy regimen may include starting the patient at a higher initial dose of the opioid agonist than would otherwise have been indicated following standard clinical guidelines. Such standard guidelines would be known to the physician and are typically based on, for example, the dosing guidelines contained in the pharmaceutical package insert and/or printed in a text such as the Physician's Desk Reference or pharmacopeia. In embodiments, a ‘higher’ dose is 10%, 20%, 30%, 40%, or 50% higher than the recommended starting dose based on clinical guidelines. Further adjustments to the patient's therapy regimen may include conjunctive therapy, such as psychosocial therapies aimed at preventing relapse, antidepressant therapy, anxiolytic therapy, and/or alternative non-opioid pain therapies.

In embodiments, a patient is identified as at high risk of non-completion if the patient's combinatorial genetic risk score as determined according to the methods described herein is statistically significant with a p-value of less than or equal to 0.05 and an odds ratio greater than or equal to 1.5. A patient is at intermediate risk if the patient's combinatorial genetic risk score as determined according to the methods described herein is statistically significant with a p-value of less than or equal to 0.05 and an odds ratio between 1 and 1.5. A patient is at low risk if none of the above conditions are met.

Also provided are methods for designing a customized therapeutic regimen for a patient that minimizes the risk of non-completion of opioid agonist replacement therapy. The methods comprise identifying the patient's risk of non-completion before the start of therapy, using a genetic screen as described herein. The methods may also comprise providing an assessment of that risk, and suggestions for interventions and/or adjustments to the patient's therapy regimen, as described above. The methods may also include generating and outputting a report identifying a patient according to risk, assessment of that risk, and suggestions for interventions and/or adjustments to the patient's therapy regimen according to the patient's relative risk.

Also provided are methods for selecting an opioid agonist medication for a patient that minimizes the patient's risk of non-completion of opioid agonist replacement therapy. The methods comprise identifying the patient's risk of non-completion before the start of therapy, using a genetic screen as described herein, and recommending a selection of methadone for the patient identified as at a high or intermediate risk of non-completion.

In embodiments, the methods described here provide an output indicating a patient's risk based upon the patient's genetic phenotype as determined from the patient's genotype or haplotype at one or more genetic variants in at least two genes, as described herein, and optionally one or more additional patient specific factors as described below. The output of risk provided by the methods described here may be referred to as the “risk assessment”. The risk assessment incorporates information about the patient's phenotype as determined by genotype at one or more genetic variants. The risk assessment may also incorporate other non-genetic information about the patient, as discussed below.

In embodiments, the patient is a human patient, and more specifically an adult patient, a pediatric patient, or an elderly patient, as those terms are understood in the medical arts. In certain embodiments, the patient is further defined according to the patient's ancestry. In embodiments the patient self-identifies or is genetically determined to be a member of a human population selected from African, North African, Southern African, European, Western European, Northern European, Asian, Japanese, Han Chinese, and Korean. In embodiments, the patient's ancestry is determined by genetic analysis according to routine methods. In embodiments, the patient's ancestry is self-reported.

### Modelling of Genetic Risk

The risk model described here incorporates information about a patient's genetic phenotype as determined by the patient's genotype and/or haplotype at one or more genetic variants in at least two genes, and provides an output in the form of a composite genetic risk score and/or risk assessment indicating the likelihood that the patient will fail to complete opioid agonist replacement therapy.

In embodiments, the risk assessment is qualitative, e.g., high, intermediate, low. In embodiments, the risk assessment is quantitative and represented by a numerical value, such as an integer. The risk assessment incorporates the patient's composite genetic risk score in the model. In embodiments, the composite genetic risk score is the sum of the genetic risk scores assigned to each gene. In embodiments, the genetic risk score for each gene is based on the genetic phenotype derived from the genotype or haplotype for that gene.

In accordance with the methods described here, a patient is identified as having a high, intermediate, or low risk of non-completion of opioid agonist replacement therapy based on the patient's composite genetic risk score. In some embodiments, where the composite genetic risk score has a p-value of less than or equal to 0.05 and an odds ratio greater than or equal to 1.5, the subject is identified as being at high risk of non-completion. In some embodiments, where the composite genetic risk score has a p-value of less than or equal to 0.05 and an odds ratio between 1 and 1.5, the subject is identified as being at intermediate risk. In some embodiments, if none of the foregoing conditions are met, the subject is considered to be at low risk. In embodiments, the assignment of high, intermediate and low risk is accomplished by means of a model. In embodiments, the model sorts patients into bins corresponding to high, intermediate and/or low risk of non-completion. In embodiments, the model has a sensitivity greater than 90, 95 or 99%. In embodiments, the model has a specificity of greater than 90, 95 or 99%. In embodiments, the positive predictive value of the model in the testing population is greater than 30, 50 or 75%. In embodiments, the negative predictive value of the model in the testing population is greater than 80, 85, 90 or 93%.

### Risk Markers and Alleles

In embodiments, the methods described here comprise assaying a biological sample from a subject at one or more genetic loci to determine the patient's genotype at one or more genetic variants within the loci. Table 1.1 provides an illustrative, non-exhaustive list of genetic variants that may be assayed for each of five pharmacokinetic (PK) genes of the cytochrome P40 family (“CYP genes”), CYP1A2, CYP2B6, CYP2C9, CYP2C19, CYP2D6, and CYP3A4, and how the genetic variants relate to enzyme activity, based upon the scientific literature. For each gene, representative star alleles are shown, along with their haplotypes (based on the genomic DNA, unless indicated as cDNA) and associated enzyme activity, where known. The haplotypes may consist of one or more single nucleotide polymorphisms (SNPs), insertions and/or deletions (InDels), as well as copy number variants (CNVs). Lists of the known star alleles for human CYP genes and their associated protein activity levels (if known) are maintained by various groups including the US National Center for Biotechnology Information and the Karolinska Institute of Sweden. Throughout this disclosure, SNPs may be referred to by their “rs” number. The “rs” number for a given SNP is a reference number provided by the HapMap consortium. The rs number is sufficient to obtain much of the known information regarding a particular SNP, for example by querying the rs number in the HapMap database or similar databases including the UCSC Genome Bioinformatics Web Page and similar databases maintained by the US National Center for Biotechnology Information.

TABLE 1.1
Exemplary Genetic Markers of CYP Genes and Associated Phenotypes.

| | Star | | Enzyme |
|---|---|---|---|
| Gene | Allele | Haplotype [genomic unless indicated as cDNA] | Activity |
| CYP1A2 | *1A | no changes [wild-type sequence] | normal |
| | *1B | 5347C > T | normal |
| | *1C | −3860G > A | decr |
| | *1D | −2467delT | incr |
| | *1F | −163C > A | incr |
| | *1K | −729C > T, −739T > G, −163C > A | decr |
| | *3 | −2116G > A | decr |
| | *4 | 2499A > T | decr |
| | *6 | 5090C > T | decr |
| | *7 | 3533G > A | decr |
| | *8 | 5166G > A | decr |
| | *11 | 558C > A | decr |
| | *15 | 125C > G | decr |
| | *16 | 2473G > A; 5347T > C | decr |
| CYP2B6 | *1 | no changes [wild-type sequence] | normal |
| | *4A-D | 785A > G (cDNA) | incr |
| | *6A-C | 516G > T; 785A > G (cDNA) | decr |
| | *9 | 516G > T (cDNA) | decr |
| CYP2C9 | *1 | no changes [wild-type sequence] | normal |
| | *2A-C | 430C > T (cDNA) | none |
| | *3A, B | 1075A > C (cDNA) | none |
| | *4 | 1076T > C (cDNA) | none |
| | *5 | 1080C > G (cDNA) | none |
| | *6 | 818delA (cDNA) | none |
| CYP2C19 | *1 | | normal |
| | *2A-H | 99C > T, 681G > A, 990C > T, 991A > G (cDNA) | none |
| | *3A-C | 636G > A, 991A > G, 1251A > C (cDNA) | none |
| | *4A, B | 1A > G, 99C > T, 991A > G (cDNA) | none |
| | *5A, B | 1297C > T (cDNA) | none |
| | *6 | 99C > T, 395G > A, 991A > G (cDNA) | none |
| | *7 | 19294T > A | none |
| | *8 | 358T > C (cDNA) | none |
| | *17 | 99C > T; 991A > G (cDNA) | incr |
| CYP2D6 | *1 | no changes [wild-type sequence] | normal |
| | *1XN | N active genes [duplication] | incr |
| | *2A-M | 2850C > T; 4180G > C | normal |
| | *2XN | 1661G > C; 2850C > T; 4180G > C, n = 2, 3, 4, 5, or 13 | incr |
| | *3A, B | 2549delA | none |
| | *4A-P | 1846G > A | none |
| | *5 | CYP2D6 deleted | none |
| | *6A-D | 1707delT | none |
| | *7 | 2935A > C | none |
| | *8 | 1661G > C; 1758G > T; 2850C > T; 4180G > C | none |
| | *9 | 2615_2617delAAG | decr |
| | *10A-D | 100C > T; 1661G > C; 4180G > C | decr |
| | *11 | 883G > C; 1661G > C; 2850C > T; 4180G > C | none |
| | *12 | 124G > A; 1661G > C; 2850C > T; 4180G > C | none |
| | *14A, B | 2850C > T; 4180G > C | none |
| | *15 | 137_138insT | none |
| | *17 | 1023C > T; 1661G > C; 2850C > T; 4180G > C | decr |
| | *41 | −1235A > G; −740C > T; −678G > A; CYP2D7 gene conversion in | decr |
| | | intron 1; 1661G > C; 2850C > T; 2988G > A; 4180G > C | |
| CYP3A4 | *1 | no changes [wild-type sequence] | normal |
| | *13 | 15389C > T | none |
| | *15A, B | 14269G > A | none |
| | *22 | 22026C > T | decr |

In the methods described here, a subject's genotype or haplotype is assigned a “genetic phenotype” and the genetic phenotype is used in the statistical analysis, for example to test the association of the phenotype with non-completion of therapy. For the CYP genes individually, metabolic phenotypes are assigned based upon the number of functional alleles that the subject has at each genetic locus, e.g., at each of loci associated with CYP1A2, CYP2B6, CYP2C9, CYP2C19, CYP2D6, and CYP3A4. The genetic loci associated with each of these genes is known in the art. By way of illustration, Table 3 below provides the genomic coordinates for each of these genes.

Table 1.2 below provides an example of how the number of functional alleles of CYP genes is associated with a metabolic phenotype selected from poor (PM), intermediate (IM), extensive (EM), and ultra-rapid (UM), based on the patient's total number of functional alleles for a given gene. These exemplary assignments are based upon a commercially available kit (AmpliChip™ by Roche). The term ‘functional’ refers to ‘normal’ enzyme activity in Table 1.1 above, ‘increased’ function is denoted ‘incr’ in the table, ‘decreased’ function is denoted ‘decr’ and the term ‘absent’ refers to alleles with no activity, denoted ‘none’ in the table.

TABLE 1.2
Exemplary Assignment of Metabolic Phenotypes Based on
Number of Functional Alleles for CYP genes.

| Alleles | Prediction | Activity Score (AS) |
|---|---|---|
| 3 or more functional alleles | UM | >2.0 |
| 1 or 2 functional alleles | EM | 1.5-2.0 |
| 1 increased function allele paired with | | |
| a decreased functional allele or absent | | |
| 1, or 2 reduced function alleles | IM | 0.5-1.0 |
| 2 absent functional alleles | PM | 0.0 |

In embodiments, the methods described here comprise assaying a biological sample from a subject for one more genetic variants in one or more pharmacodynamic (PD) genes selected from ADR2A, COMT, HTR2A, OPRM1, and SLC6A4. Table 1.3 provides the genetic variants that are assayed for each of these five pharmacodynamics genes. Table 2 below provides the reference sequences for each of these genes.

TABLE 1.3
Genetic Markers in Pharmacodynamic Genes Associated
with 24-week Non-completers.

| Gene | Genetic Variant | Genotype |
|---|---|---|
| ADRA2A | −1291C > G | CC |
| (rs1800544) | | GC |
| | | GG |
| COMT | Val158Met | Val/Val |
| (rs4680) | | Val/Met |
| | | Met/Met |
| HTR2A | −1438G > A | GG |
| (rs6311) | | AG |
| | | AA |
| OPRM1 | 118A > G | GG |
| (rs1799971) | | AG |
| | | AA |
| SLC6A4 | −2063 to −1714 | S/S |
| (5-HTTLPR) | deletion | L/S* |
| | | L/L |

*L = long, S = short

TABLE 2
Reference Sequences for PD genes

| rs number | SNP (human) | Gene |
|---|---|---|
| rs1800544 | −1291C > G | ADRA2A adrenergic receptor alpha 2A |
| rs4680 | Val158Met | COMT catechol-O-methyltransferase |
| rs6311 | −1438A > G | HTR2A 5-Hydroxytryptamine Receptor 2A |
| rs1799971 | 118A > G | OPRM1 opioid receptor mu 1 |
| | −2063 to | SLC6A4 serotonin transporter |
| | −1714 | |
| | deletion | |

TABLE 3
Reference Sequences for PK genes

| | Chromosome | | | |
|---|---|---|---|---|
| CYP Gene | No. | Human Assembly | Start | End |
| CYP1A2 | Chr15 | GRCh38/hg38 | 73,748,844 | 75,756,202 |
| CYP2B6 | Chr19 | GRCh38/hg38 | 39,991,299 | 42,018,398 |
| CYP2C9 | Chr10 | GRCh38/hg38 | 93,938,671 | 95,989,390 |
| CYP2C19 | Chr10 | GRCh38/hg38 | 93,762,624 | 95,853,260 |
| CYP2D6 | Chr22 | GRCh38/hg38 | 41,126,499 | 43,130,906 |
| CYP3A4 | Chr7 | GRCh38/hg38 | 98,756,960 | 100,784,188 |

### Methods of Treating Opioid Addiction

The methods described here provide for treating a patient with opioid agonist replacement therapy in a manner that maximizes the patient's likelihood of completing the therapy, therefore improving treatment outcome for the patient.

The methods comprise determining the patient's genotype at one or more genetic markers in a panel of at least two genes and assigning to the patient a composite genetic risk score based upon the individual risk scores for each gene, as determined according to the present methods.

In embodiments, the composite genetic risk score may optionally be combined with one or more additional patient-specific data attributes. The one or more patient-specific data attributes may be selected from the patient's ancestry, duration of addiction, baseline severity of addiction, baseline weight, age, and gender. In embodiments, the methods are applicable to a patient regardless of the patient's ancestry because the particular risk allele(s) pertinent to the patient's risk assessment occur with similar frequencies among the major human population groups.

The methods described here also provide for different treatments, or different treatment regimens, for the patient depending on the patient's risk assessment.

In embodiments, the methods further comprise one or more additional steps selected from testing the patient for one or more additional genetic markers; advising and/or counseling the patient with respect to the results of a risk assessment; transmitting, advising and/or conveying the results of an a risk assessment to a physician, medical service provider or other authorized third party; altering the patient's treatment regimen based on the results of a risk assessment in order to lower the patients risk of not completing the therapy, or any combination of the above.

Genotyping is performed by techniques known in the art, for example, PCR analysis, DNA sequencing, 5′exonuclease fluorescence assay, sequencing by probe hybridization, dot blotting, and oligonucleotide array (DNA Chip) hybridization analysis, or combinations thereof. Such techniques are described, for example, in Ausubel et al. (eds), 1989, Current Protocols in Molecular Biology, Green Publishing Associates, Inc., and John Wiley & Sons, Inc., New York, at p. 2.10.3, and in Maniatis et al., in Molecular Cloning (A Laboratory Manual), Cold Spring Harbor Laboratory, 1982, p. 387 389), and more recent versions of these or similar texts. Real-time PCR methods that can be used to detect SNPs, include, e.g., Taqman or molecular beacon-based assays (U.S. Pat. Nos. 5,210,015; 5,487,972; and PCT WO 95/13399) are useful to monitor for the presence or absence of a SNP. Genotyping technology is commercially available, for example from companies such as Applied Biosystems, Inc. (Foster City, Calif.). Any suitable biological sample from the patient can be used as the source of the DNA for genotyping.

In embodiments, a subject is treated with an opioid agonist based on the results of a model as described herein. In embodiments, a subject identified as high risk is treated as follows: administer an initial dose of buprenorphine that is higher than starting dose determined according to the clinical guidelines; or administer methadone to the patient. In embodiments, a subject identified as intermediate risk is treated as follows: administer an initial dose of buprenorphine that is higher than starting dose determined according to the clinical guidelines; or administer methadone to the patient.

### Kits

The present invention also contemplates products and kits for practicing the present methods. In embodiments, a kit comprises a set of primers adapted to amplify, in a polymerase chain reaction, a set of nucleotide sequences comprising one or more of the genetic markers described here. In embodiments, the kit comprises a set of primers adapted to amplify, in a polymerase chain reaction, at least one nucleotide sequence comprising a genetic marker selected from the group consisting of −2063 to −1714 deletion of SLC6A4 (5-HTTLPR) and COMT rs4680(Val158Met).

In embodiments, a kit comprises one or more polynucleotide probes adapted to hybridize with at least one genetic marker selected from the group consisting of −2063 to −1714 deletion of SLC6A4 (5-HTTLPR) and COMT rs4680(Val158Met).

In the context of this embodiment, the term “hybridize” refers to specific hybridization such as occurs between the probe and its complementary nucleotide sequence under high stringency hybridization conditions, as those conditions are understood in the art. In one embodiment, the probe is labeled with a detectable label, such as a radionuclide, a fluorescent molecule, a magnetic bead, or chemical entity, or any other suitable label that can be attached to or incorporated within a polynucleotide sequence. In one embodiment, the probe is attached covalently or physically associated with a support for example, but not limited to a biochip, array, slide, multi-well plate, bead or the like. In an embodiment, the probe comprises an array of nucleic acids attached or associated with a solid support.

The kits described here may also optionally comprise one or more reagents and/or products including, but not limited to, one or more buffers for performing PCR or probe hybridization, or any step in such a process as would be known to a person of skill in the art, one or more DNA amplifying enzymes, or any combination thereof; one or more reagents, components and products for genotyping the polymorphisms as described herein, including, but not limited to those used in exonuclease assays, nucleotide sequencing, or any combination thereof; one or more reagents, components or products for performing a DNA sequencing reaction that determines the sequence of a nucleotide sequence of an SNP defined herein; a gene chip or array comprising one or a plurality of nucleotide sequences defining the genetic variants described here, and; one or more sets of instructions for using the components as described herein, practicing the methods of the present invention as described herein, interpreting the data obtained from practicing the methods of the present invention or; any combination or sub-combination thereof.

Also provided are individual components of the kit, for example, but not limited to any composition described in the kit or elsewhere in the application. In a representative embodiment, the present invention provides one or more nucleic acid primers or probes.

The nucleic acid primers and probes may be of any suitable length for use in the method of the present invention. Without wishing to be limiting in any manner, it is generally preferred that the primers and probes be between about 9 and about 100 nucleotides, for example, but not limited to about 7, 8, 9, 10, 11, 12, 13, 14, 15, 16, 17, 18, 19, 20, 21, 23, 25, 27, 29, 30, 35, 40, 45, 50, 60, 70, 80, 90, about 100 nucleotides or any amount therein between. The length of the primers and probes may also be defined by a range of any two of the values provided above or any two values therein between. With respect to probes, it is generally preferred that the probe comprise at least one, more preferably 3 or more nucleotides on each side of the polymorphic site. It is also contemplated that one or more of the primers or nucleic acid probes may be labeled as is known in the art, for example, but not limited to, with a radioactive element or tag, fluorophore, or the like.

Also provided is a microarray, gene chip or the like which comprises nucleotide sequences sufficient to identify each of the genetic markers described herein. For example, the array or chip may contain sequences comprising the polymorphic site of interest. The array also may comprise the complement of the nucleotide sequences or a fragment thereof which comprises the polymorphic site. Preferably, the nucleotide sequences are of a length such as, but not limited to 7, 8, 9, 10, 11, 12, 13, 14, 15, 16, 17, 18, 19, 20, 21, 22, 23, 24, 25 or more continuous nucleotides to permit strong hybridization under stringent hybridization conditions. In a preferred embodiment the array comprises or consists of one or more nucleotide sequences comprising the polymorphic sites of one or more of ADRA2A, COMT, HTR2A, OPRM1, SLC6A4, CYP1A, CYP2B6, CYP2C9, CYP2C19, and CYP2D6, as described herein.

One or more aspects or features of the subject matter described herein can be realized in digital electronic circuitry, integrated circuitry, specially designed application specific integrated circuits (ASICs), field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, modules, model generators, computer instructions, software, software applications, applications, components, or code, include machine instructions for a programmable processor, and can be implemented in a high-level procedural language, an object-oriented programming language, a functional programming language, a logical programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example as would a processor cache or other random access memory associated with one or more physical processor cores.

To provide for interaction with a user, one or more aspects or features of the patient matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including, but not limited to, acoustic, speech, or tactile input. Other possible input devices include, but are not limited to, touch screens or other touch-sensitive devices such as single or multi-point resistive or capacitive trackpads, voice recognition hardware and software, optical scanners, optical pointers, digital image capture devices and associated interpretation software, and the like.

### Medicaments

The present invention also contemplates medicaments for use in patients identified by the present methods, models and kits. In embodiments, the medicaments are buprenorphine, methodone, or buprenorphine/methadone (SUBOXONE).

In embodiments, this disclosure provides for methadone for use in treating opioid addiction in a patient, comprising assaying a sample from the patient, determining if the patient has a composite genetic risk score indicating a high risk of non-completion of opioid agonist replacement therapy, and administering a therapeutically effective amount of methadone to the patient if a composite genetic risk score indicating a high risk of non-completion of opioid agonist replacement therapy is present.

In embodiments, the disclosure provides for buprenorphine for use in treating opioid addiction in a patient, comprising assaying a sample from the patient, determining if the patient has a composite genetic risk score indicating a high risk of non-completion of opioid agonist replacement therapy, and administering to the patient an initial dose of buprenorphine that is higher than starting dose determined according to the clinical guidelines if a composite genetic risk score indicating a high risk of non-completion of opioid agonist replacement therapy is present.

In embodiments, the medicament is provided for use, wherein determining if the patient has a composite genetic risk score indicating a high risk of non-completion of opioid agonist replacement therapy comprises determining, in an ex vivo sample from the subject, a genetic phenotype for one or more pharmacodynamic genes, and optionally determining the subject's genetic phenotype for one or more cytochrome P450 (CYP) genes, and generating a composite genetic risk score for the subject.

In embodiments, the medicament is provided for use, further comprising, identifying the subject as having a high, risk of non-completion of opioid agonist replacement therapy based on the subject's composite genetic risk score.

In embodiments, the pharmacodynamics genes comprise or are selected from the genes and panels disclosed herein. In embodiments, the CYP genes comprise or are selected from the genes and panels disclosed herein.

In embodiments, methadone is provided for use, wherein determining the subject's genetic phenotype for one or more cytochrome P450 (CYP) genes comprises combining combinatorial pharmacogenetics with a physiological algorithm for predicting medication exposure.

In embodiments, methadone is provided for use, wherein determining the subject's genetic phenotype for one or more pharmacodynamic genes comprises assigning a numerical score to each genotype associated with a pharmacodynamic gene based on the difference between the completion percentage for that genotype and the mean completion percentage in a reference population.

The present invention will be further illustrated in the following additional embodiments.

### LISTING OF ADDITIONAL EMBODIMENTS

1. A method of identifying a subject at risk of non-completion of opioid agonist replacement therapy, the method comprising

determining the subject's genetic phenotype for one or more pharmacodynamic genes, and optionally determining the subject's genetic phenotype for one or more cytochrome P450 (CYP) genes,

generating a composite genetic risk score for the subject, and

identifying the subject as having a high, intermediate, or low risk of non-completion of opioid agonist replacement therapy based on the subject's composite genetic risk score.

2. A method of treating opioid addiction in a human subject in need thereof, the method comprising

determining the subject's genetic phenotype for one or more pharmacodynamic genes, and optionally determining the subject's genetic phenotype for one or more cytochrome P450 (CYP) genes,

generating a composite genetic risk score for the subject,

identifying the subject as having a high, intermediate, or low risk of non-completion of opioid agonist replacement therapy based on the subject's composite genetic risk score, and

administering an opioid agonist to the subject who is identified as being at high or intermediate risk of non-completion as follows:
administer an initial dose of buprenorphine that is higher than starting dose determined according to the clinical guidelines; or
administer methadone to the patient.
   3. A method of treating opioid addiction in a human subject in need thereof, the method comprising

determining the subject's genetic phenotype for one or more pharmacodynamic genes, and optionally determining the subject's genetic phenotype for one or more cytochrome P450 (CYP) genes,

generating a composite genetic risk score for the subject,

identifying the subject as having a high, intermediate, or low risk of non-completion of opioid agonist replacement therapy based on the subject's composite genetic risk score, and

administering an opioid agonist to the subject who is identified as being at high risk of non-completion as follows:
administer an initial dose of buprenorphine that is higher than starting dose determined according to the clinical guidelines; or
administer methadone to the patient.
   4. A method of treating opioid addiction in a human subject in need thereof, the method comprising

determining the subject's genetic phenotype for one or more pharmacodynamic genes, and optionally determining the subject's genetic phenotype for one or more cytochrome P450 (CYP) genes,

generating a composite genetic risk score for the subject,

identifying the subject as having a high, intermediate, or low risk of non-completion of opioid agonist replacement therapy based on the subject's composite genetic risk score, and

administering an opioid agonist to the subject who is identified as being at intermediate risk of non-completion as follows:
administer an initial dose of buprenorphine that is higher than starting dose determined according to the clinical guidelines; or
administer methadone to the patient.
   5. An in vitro method of identifying a subject at risk of non-completion of opioid agonist replacement therapy, comprising

determining, in an ex vivo sample from the subject, a genetic phenotype for one or more pharmacodynamic genes, and optionally determining the subject's genetic phenotype for one or more cytochrome P450 (CYP) genes,

generating a composite genetic risk score for the subject, and

identifying the subject as having a high, intermediate, or low risk of non-completion of opioid agonist replacement therapy based on the subject's composite genetic risk score.

6. The method of any of embodiments 1-5, wherein the pharmacodynamic genes are comprised of one or more of ADRA2A, COMT, HTR2A, OPRM1, and SLC6A4.
7. The method of any of embodiments 1-5, wherein the pharmacodynamic genes are selected from one or more of ADRA2A, COMT, HTR2A, OPRM1, and SLC6A4.
8. The method of any of embodiments 1-7, wherein the cytochrome P450 genes are comprised of one or more of CYP1A, CYP2B6, CYP2C9, CYP2C19, and CYP2D6.
9. The method of any of embodiments 1-7, wherein the cytochrome P450 genes are selected from one or more of CYP1A, CYP2B6, CYP2C9, CYP2C19, and CYP2D6.
10. The method of any of embodiments 1-9, wherein the composite genetic risk score for the subject comprises the sum of the risks associated with each genetic phenotype.
11. The method of any of embodiments 1-10, wherein the genetic phenotype of each of the one or more CYP genes is a combination phenotype based upon the number of functional alleles at the genetic locus.
12. The method of any of embodiments 1-11, wherein the subject's genetic phenotype is determined for a panel of two genes selected from the group consisting of

COMT, CYP3A4;

COMT, HTR2A;

COMT, SLC6A4;

HTR2A, CYP3A4;

SLC6A4, HTR2A; and

SLC6A4, CYP3A4.

13. The method of any of embodiments 1-11, wherein the subject's genetic phenotype is determined for a panel of genes selected from the group consisting of

COMT, HTR2A, CYP3A4;

COMT, SLC6A4, CYP3A4;

COMT, SLC6A4, HTR2A; and

SLC6A4, HTR2A, CYP3A4.

14. The method of any of embodiments 1-11, wherein the subject's genetic phenotype is determined for a panel of genes selected from the group consisting of

COMT, SLC6A4, HTR2A, CYP3A4;

COMT, SLC6A4, HTR2A, CYP3A4, CYP2B6;

COMT, SLC6A4, HTR2A, CYP3A4, CYP2C19;

COMT, SLC6A4, HTR2A, CYP3A4, CYP1A2;

COMT, SLC6A4, HTR2A, CYP3A4, CYP2C9; and

COMT, SLC6A4, HTR2A, CYP3A4, CYP2D6.

15. The method of any of embodiments 1-11, wherein the subject's genetic phenotype is determined for a panel of genes selected from the group consisting of

COMT, SLC6A4, HTR2A, CYP3A4, CYP2B6, CYP2C19;

COMT, SLC6A4, HTR2A, CYP3A4, CYP2B6, CYP1A2;

COMT, SLC6A4, HTR2A, CYP3A4, CYP2B6, CYP2C9;

COMT, SLC6A4, HTR2A, CYP3A4, CYP2B6, CYP2D6;

COMT, SLC6A4, HTR2A, CYP3A4, CYP2C19, CYP1A2;

COMT, SLC6A4, HTR2A, CYP3A4, CYP2C19, CYP2C9; and

COMT, SLC6A4, HTR2A, CYP3A4, CYP2C19, CYP2D6.

16. The method of any of embodiments 1-11, wherein the subject's genetic phenotype is determined for a panel of genes selected from the group consisting of

COMT, SLC6A4, HTR2A, CYP3A4, CYP2B6, CYP2C9, CYP2D6;

COMT, SLC6A4, HTR2A, CYP3A4, CYP2B6, CYP2C9, CYP2D6, CYP2C19, CYP1A2.

17. The method of any of embodiments 1-11, wherein the genetic phenotype is determined by assaying for one or more of the following genetic variants in the one or more pharmacodynamic genes: ADRA2A (rs1800544), COMT (rs4680), HTR2A (rs6311), OPRM1 (rs1799971), SLC6A4 (5-HTTLPR).
18. The method of any of embodiments 1-11, wherein the one or more pharmacodynamic genes includes COMT and SLC6A4.
19. The method of any of embodiments 1-18, wherein the genetic phenotype is assigned based on the genotype at each genetic variant.
20. The method of any of embodiment 1-19, wherein determining the subject's genetic phenotype for one or more cytochrome P450 (CYP) genes comprises
combining combinatorial pharmacogenetics with a physiological algorithm for predicting medication exposure.
   21. The method of any of embodiment 1-19, wherein determining the subject's genetic phenotype for one or more pharmacodynamic genes comprises
assigning a numerical score to each genotype associated with a pharmacodynamic gene based on the difference between the completion percentage for that genotype and the mean completion percentage in a reference population.
   22. The method of any of embodiment 1-21, further comprising identifying the subject as having a high, intermediate, or low risk of non-completion of opioid agonist replacement therapy based on the subject's composite genetic risk score as follows
if the composite genetic risk score has a p-value of less than or equal to 0.05 and an odds ratio greater than or equal to 1.5, the subject is at high risk;
if the composite genetic risk score has a p-value of less than or equal to 0.05 and an odds ratio between 1 and 1.5, the subject is at intermediate risk; and
if none of the above conditions are met, the subject is at low risk.
   23. Methadone for use in treating opioid addiction in a patient, comprising assaying a sample from the patient,

determining if the patient has a composite genetic risk score indicating a high risk of non-completion of opioid agonist replacement therapy, and

administering a therapeutically effective amount of methadone to the patient if a composite genetic risk score indicating a high risk of non-completion of opioid agonist replacement therapy is present.

24. Methadone according to the use of embodiment 23, wherein determining if the patient has a composite genetic risk score indicating a high risk of non-completion of opioid agonist replacement therapy comprises

determining, in an ex vivo sample from the subject, a genetic phenotype for one or more pharmacodynamic genes, and optionally determining the subject's genetic phenotype for one or more cytochrome P450 (CYP) genes, and

generating a composite genetic risk score for the subject.

25. Methadone according to the use of embodiment 24, further comprising,
identifying the subject as having a high, risk of non-completion of opioid agonist replacement therapy based on the subject's composite genetic risk score.
   26. Methadone according to the use of any of embodiments 24-25, wherein the pharmacodynamic genes are comprised of one or more of ADRA2A, COMT, HTR2A, OPRM1, and SLC6A4.
   27. Methadone according to the use of any of embodiments 24-25, wherein the pharmacodynamic genes are selected from one or more of ADRA2A, COMT, HTR2A, OPRM1, and SLC6A4.
   28. Methadone according to the use of any of embodiments 24-27, wherein the cytochrome P450 genes are comprised of one or more of CYP1A, CYP2B6, CYP2C9, CYP2C19, and CYP2D6.
   29. Methadone according to the use of any of embodiments 24-27, wherein the cytochrome P450 genes are selected from one or more of CYP1A, CYP2B6, CYP2C9, CYP2C19, and CYP2D6.
   30. Methadone according to the use of any of embodiments 24-29, wherein the composite genetic risk score for the subject comprises the sum of the risks associated with each genetic phenotype.
   31. Methadone according to the use of any of embodiments 24-30, wherein the genetic phenotype of each of the one or more CYP genes is a combination phenotype based upon the number of functional alleles at the genetic locus.
   32. Methadone according to the use of any of embodiments 24-31, wherein the subject's genetic phenotype is determined for a panel of two genes selected from the group consisting of

COMT, CYP3A4;

COMT, HTR2A;

COMT, SLC6A4;

HTR2A, CYP3A4;

SLC6A4, HTR2A; and

SLC6A4, CYP3A4.

33. Methadone according to the use of any of embodiments 24-31, wherein the subject's genetic phenotype is determined for a panel of genes selected from the group consisting of

COMT, HTR2A, CYP3A4;

COMT, SLC6A4, CYP3A4;

COMT, SLC6A4, HTR2A; and

SLC6A4, HTR2A, CYP3A4.

34. Methadone according to the use of any of embodiments 24-31, wherein the subject's genetic phenotype is determined for a panel of genes selected from the group consisting of

COMT, SLC6A4, HTR2A, CYP3A4;

COMT, SLC6A4, HTR2A, CYP3A4, CYP2B6;

COMT, SLC6A4, HTR2A, CYP3A4, CYP2C19;

COMT, SLC6A4, HTR2A, CYP3A4, CYP1A2;

COMT, SLC6A4, HTR2A, CYP3A4, CYP2C9; and

COMT, SLC6A4, HTR2A, CYP3A4, CYP2D6.

35. Methadone according to the use of any of embodiments 24-31, wherein the subject's genetic phenotype is determined for a panel of genes selected from the group consisting of

COMT, SLC6A4, HTR2A, CYP3A4, CYP2B6, CYP2C19;

COMT, SLC6A4, HTR2A, CYP3A4, CYP2B6, CYP1A2;

COMT, SLC6A4, HTR2A, CYP3A4, CYP2B6, CYP2C9;

COMT, SLC6A4, HTR2A, CYP3A4, CYP2B6, CYP2D6;

COMT, SLC6A4, HTR2A, CYP3A4, CYP2C19, CYP1A2;

COMT, SLC6A4, HTR2A, CYP3A4, CYP2C19, CYP2C9; and

COMT, SLC6A4, HTR2A, CYP3A4, CYP2C19, CYP2D6.

36. Methadone according to the use of any of embodiments 24-31, wherein the subject's genetic phenotype is determined for a panel of genes selected from the group consisting of

COMT, SLC6A4, HTR2A, CYP3A4, CYP2B6, CYP2C9, CYP2D6;

COMT, SLC6A4, HTR2A, CYP3A4, CYP2B6, CYP2C9, CYP2D6, CYP2C19, CYP1A2.

37. Methadone according to the use of any of embodiments 24-31, wherein the genetic phenotype is determined by assaying for one or more of the following genetic variants in the one or more pharmacodynamic genes: ADRA2A (rs1800544), COMT (rs4680), HTR2A (rs6311), OPRM1 (rs1799971), SLC6A4 (5-HTTLPR).
38. Methadone according to the use of any of embodiments 24-31, wherein the one or more pharmacodynamic genes includes COMT and SLC6A4.
39. Methadone according to the use of any of embodiments 24-38, wherein the genetic phenotype is assigned based on the genotype at each genetic variant.
40. Methadone according to the use of embodiment 24-39, wherein determining the subject's genetic phenotype for one or more cytochrome P450 (CYP) genes comprises
combining combinatorial pharmacogenetics with a physiological algorithm for predicting medication exposure.
   41. Methadone according to the use of embodiment 24-39, wherein determining the subject's genetic phenotype for one or more pharmacodynamic genes comprises
assigning a numerical score to each genotype associated with a pharmacodynamic gene based on the difference between the completion percentage for that genotype and the mean completion percentage in a reference population.
   42. Buprenorphine for use in treating opioid addiction in a patient, comprising

assaying a sample from the patient,

determining if the patient has a composite genetic risk score indicating a high risk of non-completion of opioid agonist replacement therapy, and

administering to the patient an initial dose of buprenorphine that is higher than starting dose determined according to the clinical guidelines if a composite genetic risk score indicating a high risk of non-completion of opioid agonist replacement therapy is present.

43 Buprenorphine according to the use of embodiment 42, wherein determining if the patient has a composite genetic risk score indicating a high risk of non-completion of opioid agonist replacement therapy comprises

determining, in an ex vivo sample from the subject, a genetic phenotype for one or more pharmacodynamic genes, and optionally determining the subject's genetic phenotype for one or more cytochrome P450 (CYP) genes, and

generating a composite genetic risk score for the subject.

44 Buprenorphine according to the use of embodiment 42, further comprising,
identifying the subject as having a high, risk of non-completion of opioid agonist replacement therapy based on the subject's composite genetic risk score.
   45. Buprenorphine according to the use of any of embodiments 42-44, wherein the pharmacodynamic genes are comprised of one or more of ADRA2A, COMT, HTR2A, OPRM1, and SLC6A4.
   46. Buprenorphine according to the use of any of embodiments 42-44, wherein the pharmacodynamic genes are selected from one or more of ADRA2A, COMT, HTR2A, OPRM1, and SLC6A4.
   47. Buprenorphine according to the use of any of embodiments 42-46, wherein the cytochrome P450 genes are comprised of one or more of CYP1A, CYP2B6, CYP2C9, CYP2C19, and CYP2D6.
   48. Buprenorphine according to the use of any of embodiments 42-46, wherein the cytochrome P450 genes are selected from one or more of CYP1A, CYP2B6, CYP2C9, CYP2C19, and CYP2D6.
   49. Buprenorphine according to the use of any of embodiments 42-48, wherein the composite genetic risk score for the subject comprises the sum of the risks associated with each genetic phenotype.
   50. Buprenorphine according to the use of any of embodiments 42-49, wherein the genetic phenotype of each of the one or more CYP genes is a combination phenotype based upon the number of functional alleles at the genetic locus.
   51. Buprenorphine according to the use of any of embodiments 42-50, wherein the subject's genetic phenotype is determined for a panel of two genes selected from the group consisting of

COMT, CYP3A4;

COMT, HTR2A;

COMT, SLC6A4;

HTR2A, CYP3A4;

SLC6A4, HTR2A; and

SLC6A4, CYP3A4.

52. Buprenorphine according to the use of any of embodiments 42-50, wherein the subject's genetic phenotype is determined for a panel of genes selected from the group consisting of

COMT, HTR2A, CYP3A4;

COMT, SLC6A4, CYP3A4;

COMT, SLC6A4, HTR2A; and

SLC6A4, HTR2A, CYP3A4.

53. Buprenorphine according to the use of any of embodiments 42-50, wherein the subject's genetic phenotype is determined for a panel of genes selected from the group consisting of

COMT, SLC6A4, HTR2A, CYP3A4;

COMT, SLC6A4, HTR2A, CYP3A4, CYP2B6;

COMT, SLC6A4, HTR2A, CYP3A4, CYP2C19;

COMT, SLC6A4, HTR2A, CYP3A4, CYP1A2;

COMT, SLC6A4, HTR2A, CYP3A4, CYP2C9; and

COMT, SLC6A4, HTR2A, CYP3A4, CYP2D6.

54. Buprenorphine according to the use of any of embodiments 42-50, wherein the subject's genetic phenotype is determined for a panel of genes selected from the group consisting of

COMT, SLC6A4, HTR2A, CYP3A4, CYP2B6, CYP2C19;

COMT, SLC6A4, HTR2A, CYP3A4, CYP2B6, CYP1A2;

COMT, SLC6A4, HTR2A, CYP3A4, CYP2B6, CYP2C9;

COMT, SLC6A4, HTR2A, CYP3A4, CYP2B6, CYP2D6;

COMT, SLC6A4, HTR2A, CYP3A4, CYP2C19, CYP1A2;

COMT, SLC6A4, HTR2A, CYP3A4, CYP2C19, CYP2C9; and

COMT, SLC6A4, HTR2A, CYP3A4, CYP2C19, CYP2D6.

55. Buprenorphine according to the use of any of embodiments 42-50, wherein the subject's genetic phenotype is determined for a panel of genes selected from the group consisting of

COMT, SLC6A4, HTR2A, CYP3A4, CYP2B6, CYP2C9, CYP2D6;

COMT, SLC6A4, HTR2A, CYP3A4, CYP2B6, CYP2C9, CYP2D6, CYP2C19, CYP1A2.

56. Buprenorphine according to the use of any of embodiments 42-50, wherein the genetic phenotype is determined by assaying for one or more of the following genetic variants in the one or more pharmacodynamic genes: ADRA2A (rs1800544), COMT (rs4680), HTR2A (rs6501), OPRM1 (rs1799971), SLC6A4 (5-HTTLPR).
57. Buprenorphine according to the use of any of embodiments 42-50, wherein the one or more pharmacodynamic genes includes COMT and SLC6A4.
58. Buprenorphine according to the use of any of embodiments 42-57, wherein the genetic phenotype is assigned based on the genotype at each genetic variant.
59. Buprenorphine according to the use of embodiment 42-58, wherein determining the subject's genetic phenotype for one or more cytochrome P450 (CYP) genes comprises
combining combinatorial pharmacogenetics with a physiological algorithm for predicting medication exposure.
   60. Buprenorphine according to the use of embodiment 42-58, wherein determining the subject's genetic phenotype for one or more pharmacodynamic genes comprises
assigning a numerical score to each genotype associated with a pharmacodynamic gene based on the difference between the completion percentage for that genotype and the mean completion percentage in a reference population.

The present invention will be further illustrated in the following examples.

### Example 1

The aim of the present study was to identify genetic markers which either alone or in combination can predict a patient's likelihood of not completing a standard treatment of opioid agonist replacement therapy. The identification of a genetic marker, or combination of markers, that is predictive of whether a patient will be a ‘completer’ or ‘non-completer’ in the context of this therapy would be useful, for example, as a means to target at risk patients for alternative treatments and/or early interventions in order to minimize risk of non-completion, increase therapy retention, and thereby improve therapy outcomes.

The present study was conducted using a dataset obtained during the ‘Starting Treatment with Agonist Replacement Therapy’ (START) trial, a 24-week, randomized, open-label, outpatient-based (9-sites) study assessing changes in liver enzymes related to treatment with methadone compared to changes in liver enzymes related to treatment with a buprenorphine/naloxone (“SUBOXONE™”) combination (n=764). The START trial also collected genetic material and other data on treatment response (see also Background section, supra).

In the present study, six pharmacokinetic genes (CYP1A2, CYP2B6, CYP2C9, CYP2C19, CYP2D6, & CYP3A4) and five pharmacodynamic genes (ADRA2A, COMT, HTR2A, OPRM1, & SLC6A4) were chosen for analysis based upon the inventors' unpublished data indicating that this combination of pharmacokinetic (PK) and pharmacodynamics (PD) genes represents a substantial contribution to the genetic component of response to opioid medications.

### Single-Gene Modeling

These 11 genes were first statistically evaluated on an individual basis for impact on completion of either the methadone treatment regimen (MET) or the buprenorphine (given in the form of buprenorphine/naloxone as noted above) treatment regimen (BUP). Completion of each treatment regimen was based on a 24 week course of treatment. Patients were considered ‘completers’ if they completed the entire 24 weeks of treatment. If a patient dropped out of treatment at any time during the 24 weeks, the patient was considered a ‘non-completer’. Thus, the outcome evaluated was binary: completion versus non-completion. Drop-out rates were not considered in this analysis.

The analysis also accounted for drug dose as follows. Drug doses were divided into 5 categories as shown in Table 4.1 below. Dose was used as a covariate in the analyses with subjects binned into each of the 5 categories according to mean dose.

TABLE 4.1
Drug Dose Bins

| Category | MET mg | BUP mg |
|---|---|---|
| 1 | 0-40 | 0-10 |
| 2 | 41-60 | 12-14 |
| 3 | 61-80 | 16-20 |
| 4 | 81-120 | 22-28 |
| 5 | 121 and higher | 30-32 |

For this analysis, the patient's genotype was evaluated at the genetic markers listed in Table 1 and Table 1.3. Genotypes and/or haplotypes were converted to phenotypes.

Logistic regression was used to model non-completion as a function of phenotype and dose (using the dose categories shown in Table 4.1) for each of the 11 genes. The analysis was conducted using each treatment arm separately and also using the combination of both treatment arms.

None of the genes had a significant effect on whether or not a patient completed the therapeutic regimen of drug treatment when the treatment arms were analyzed individually. But four genes, SLC6A4, COMT, HTR2A and CYP3A4 had significant or very close to significant impact (directional) when both treatment arms were combined. Specifically, SLC6A4 had significant effects (p-value <0.05) and COMT, HTR2A, and CYP3A4 phenotypes had directional effects (p-value <0.10). The results are shown in Tables 4.2 and 4.3 below.

TABLE 4.2
Single Gene Association with Non-Completion
(showing overall test p-value for each gene)

| | MET | BUP | COMBINED |
|---|---|---|---|
| GENE | p-value | p-value | p-value |
| CYP1A2 | 0.848 | 0.294 | 0.498 |
| CYP2B6 | 0.231 | 0.439 | 0.727 |
| CYP2C9 | 0.682 | 0.176 | 0.496 |
| CYP2C19 | 0.867 | 0.717 | 0.803 |
| CYP2D6 | 0.798 | 0.276 | 0.383 |
| CYP3A4 | 0.427 | 0.147 | 0.085 |
| ADRA2A | 0.763 | 0.964 | 0.802 |
| COMT | 0.099 | 0.524 | 0.091 |
| HTR2A | 0.422 | 0.221 | 0.079 |
| OPRM1 | 0.444 | 0.907 | 0.601 |
| SLC6A4 | 0.089 | 0.269 | 0.027 |

TABLE 4.3
Single Gene Phenotypes Associated with Non-Completion. “OR” represents odds ratio.

| | | Both Medications | | Methadone | | Suboxone | |
|---|---|---|---|---|---|---|---|
| Gene | Comparison | p-value | OR (95% CI) | p-value | OR (95% CI) | p-value | OR (95% CI) |
| CYP3A4 | EM vs IM + PM | 0.085 | 1.95 (0.91-4.19) | 0.427 | 1.66 (0.48-5.77) | 0.147 | 2.06 (0.78-5.44) |
| COMT | VAL/VAL vs VAL/MET | 0.192 | 1.32 (0.87-2) | 0.713 | 1.14 (0.56-2.34) | 0.300 | 1.32 (0.78-2.23) |
| | VAL/VAL vs MET/MET | 0.031 | 1.82 (1.06-3.15) | 0.037 | 3.16 (1.07-9.31) | 0.377 | 1.34 (0.7-2.58) |
| | VAL/MET vs MET/MET | 0.223 | 1.38 (0.82-2.32) | 0.051 | 2.76 (0.99-7.68) | 0.959 | 1.02 (0.54-1.9) |
| SLC6A4 | L/S vs L/L | 0.168 | 0.74 (0.48-1.14) | 0.503 | 0.77 (0.36-1.65) | 0.287 | 0.75 (0.44-1.27) |
| | L/S vs S/S | 0.008 | 0.52 (0.32-0.84) | 0.029 | 0.41 (0.19-0.91) | 0.121 | 0.61 (0.33-1.14) |
| | L/L vs S/S | 0.161 | 0.7 (0.43-1.15) | 0.143 | 0.54 (0.23-1.23) | 0.533 | 0.82 (0.44-1.53) |
| HTR2A | Intermediate vs Normal | 0.029 | 0.57 (0.35-0.94) | 0.213 | 0.58 (0.25-1.36) | 0.083 | 0.57 (0.3-1.08) |
| | Intermediate vs Reduced | 0.196 | 0.76 (0.5-1.15) | 0.404 | 0.73 (0.35-1.52) | 0.608 | 0.87 (0.51-1.48) |
| | Normal vs Reduced | 0.291 | 1.32 (0.79-2.23) | 0.621 | 1.26 (0.51-3.09) | 0.204 | 1.53 (0.79-2.96) |

The tables below show detail for the single gene modeling. The effects of phenotype or genotype of each gene were analyzed for methadone only, Suboxone™ only, and both medications combined. Dose-bin-category was used as a covariate in all non-completer analyses with subjects binned into quintiles by mean dose. Logistic regression was used to model non-completion as a function of phenotype for each pharmacokinetic gene.

Table 5 lists the p-value and significant status for each of the eleven genes and the covariate Dose-bin-Category for Methadone. Dose-bin-category was significant for all eleven genes. Table 6 lists the odds ratios for Methadone. For COMT, patients with a high phenotype were 3.16 times more likely not to complete the 24-week treatment compared to those with a reduced phenotype. For SLC6A4, patients with a reduced phenotype were 2.41 times more likely not to complete the 24-week treatment compared to those with an intermediate phenotype.

TABLE 5
Percentage Non-Completers Phenotype Overall Test for METHADONE.
In the “Sign” column, 1 indicates significant (p-value ≤0.05);
0 indicates not significant (p-value >0.05).

| Gene | Factor | p-value | Sign. |
|---|---|---|---|
| ADRA2A | ADRA2A_Phenotype | 0.7632 | 0 |
| | Dose_binC | 0.0144 | 1 |
| COMT | COMT_Phenotype | 0.0986 | 0 |
| | Dose_binC | 0.0099 | 1 |
| CYP1A2B | CYP1A2B_phenotype | 0.8478 | 0 |
| | Dose_binC | 0.0148 | 1 |
| CYP2B6 | CYP2B6_Phenotype | 0.2312 | 0 |
| | Dose_binC | 0.0147 | 1 |
| CYP2C19 | CYP2C19_Phenotype | 0.8665 | 0 |
| | Dose_binC | 0.0154 | 1 |
| CYP2C9 | CYP2C9_Phenotype | 0.6822 | 0 |
| | Dose_binC | 0.0173 | 1 |
| CYP2D6 | CYP2D6_Phenotype | 0.7975 | 0 |
| | Dose_binC | 0.0144 | 1 |
| CYP3A4B | Cyp3A4B_Phenotype | 0.4267 | 0 |
| | Dose_binC | 0.0163 | 1 |
| HTR2A | HTR2A_Phenotype | 0.4222 | 0 |
| | Dose_binC | 0.0171 | 1 |
| OPRM1B | OPRM1B_phenotype | 0.4444 | 0 |
| | Dose_binC | 0.0126 | 1 |
| SLC6A4 | SLC6A4_Phenotype | 0.0891 | 0 |
| | Dose_binC | 0.0198 | 1 |

TABLE 6
Percentage Non-Completers Phenotype for METHADONE; Higher Odds Ratio
indicates that patient is more likely to drop out. In the “Sign” column, 1 indicates significant (p-
value ≤ 0.05); 0 indicates not significant (p-value > 0.05).

| | | | | | 95% | 95% | |
|---|---|---|---|---|---|---|---|
| | | | | Odds | Lower | Upper | |
| Gene | Phenotype | _Phenotype | p-value | Ratio | OR | OR | Sign. |
| ADRA2A | REDUCED | TYPICAL | 0.7632 | 1.103 | 0.583 | 2.086 | 0 |
| COMT | HIGH | INTERMEDIATE | 0.7133 | 1.143 | 0.560 | 2.335 | 0 |
| | HIGH | REDUCED | 0.0370 | 3.158 | 1.072 | 9.309 | 1 |
| | INTERMEDIATE | REDUCED | 0.0514 | 2.763 | 0.994 | 7.680 | 0 |
| CYP1A2B | EM + IM | ULTRARAPID | 0.8478 | 1.064 | 0.562 | 2.015 | 0 |
| CYP2B6 | EXTENSIVE | INTERMEDIATE | 0.6073 | 1.210 | 0.585 | 2.505 | 0 |
| | EXTENSIVE | POOR | 0.2117 | 0.515 | 0.182 | 1.459 | 0 |
| | EXTENSIVE | ULTRARAPID | 0.1732 | 0.370 | 0.088 | 1.547 | 0 |
| | INTERMEDIATE | POOR | 0.1262 | 0.425 | 0.142 | 1.272 | 0 |
| | INTERMEDIATE | ULTRARAPID | 0.1119 | 0.306 | 0.071 | 1.318 | 0 |
| | POOR | ULTRARAPID | 0.6955 | 0.719 | 0.137 | 3.759 | 0 |
| CYP2C19 | EXTENSIVE | INTERMEDIATE | 0.6705 | 1.190 | 0.535 | 2.647 | 0 |
| | EXTENSIVE | POOR | 0.6942 | 0.721 | 0.141 | 3.686 | 0 |
| | EXTENSIVE | ULTRARAPID | 0.5413 | 1.919 | 0.237 | 15.529 | 0 |
| | INTERMEDIATE | POOR | 0.5718 | 0.606 | 0.107 | 3.440 | 0 |
| | INTERMEDIATE | ULTRARAPID | 0.6667 | 1.613 | 0.183 | 14.201 | 0 |
| | POOR | ULTRARAPID | 0.4599 | 2.662 | 0.198 | 35.707 | 0 |
| CYP2C9 | EXTENSIVE | INTERMEDIATE | 0.4149 | 1.374 | 0.640 | 2.951 | 0 |
| | EXTENSIVE | POOR | 0.8336 | 0.845 | 0.176 | 4.051 | 0 |
| | INTERMEDIATE | POOR | 0.5648 | 0.615 | 0.118 | 3.215 | 0 |
| CYP2D6 | EXTENSIVE | INTERMEDIATE | 0.5327 | 1.302 | 0.569 | 2.980 | 0 |
| | EXTENSIVE | POOR | 0.5522 | 0.765 | 0.316 | 1.851 | 0 |
| | EXTENSIVE | ULTRARAPID | 0.9414 | 0.952 | 0.255 | 3.552 | 0 |
| | INTERMEDIATE | POOR | 0.3163 | 0.588 | 0.208 | 1.663 | 0 |
| | INTERMEDIATE | ULTRARAPID | 0.6682 | 0.731 | 0.175 | 3.060 | 0 |
| | POOR | ULTRARAPID | 0.7698 | 1.245 | 0.288 | 5.386 | 0 |
| CYP3A4B | EM | IM + PM | 0.4267 | 1.658 | 0.477 | 5.770 | 0 |
| HTR2A | INTERMEDIATE | NORMAL | 0.2132 | 0.583 | 0.250 | 1.363 | 0 |
| | INTERMEDIATE | REDUCED | 0.4040 | 0.732 | 0.352 | 1.523 | 0 |
| | NORMAL | REDUCED | 0.6212 | 1.255 | 0.509 | 3.094 | 0 |
| OPRM1B | INT + REDUCED | NORMAL | 0.4444 | 1.355 | 0.622 | 2.952 | 0 |
| SLC6A4 | INTERMEDIATE | NORMAL | 0.5033 | 0.771 | 0.360 | 1.652 | 0 |
| | INTERMEDIATE | REDUCED | 0.0293 | 0.414 | 0.187 | 0.915 | 1 |
| | NORMAL | REDUCED | 0.1431 | 0.537 | 0.234 | 1.234 | 0 |

Table 7 lists the p-value and significance status of phenotype and drug dose-bin-category and Table 8 lists the odds ratio and significance level for phenotypes for Suboxone. Dose-bin-category was not significant for any genes. There were no significant differences observed among phenotypes for any genes.

TABLE 7
Percentage Non-Completers Phenotype Overall Test for SUBOXONE.
In the “Sign” column, 1 indicates significant (p-value ≤0.05).
0 indicates not significant (p-value >0.05);

| Gene | Factor | p-value | Sign. |
|---|---|---|---|
| ADRA2A | ADRA2A_Phenotype | 0.9638 | 0 |
| | Dose_binC | 0.4421 | 0 |
| COMT | COMT_Phenotype | 0.5241 | 0 |
| | Dose_binC | 0.4864 | 0 |
| CYP1A2B | CYP1A2B_phenotype | 0.2939 | 0 |
| | Dose_binC | 0.4487 | 0 |
| CYP2B6 | CYP2B6_Phenotype | 0.4394 | 0 |
| | Dose_binC | 0.4193 | 0 |
| CYP2C19 | CYP2C19_Phenotype | 0.7173 | 0 |
| | Dose_binC | 0.4808 | 0 |
| CYP2C9 | CYP2C9_Phenotype | 0.1758 | 0 |
| | Dose_binC | 0.4759 | 0 |
| CYP2D6 | CYP2D6_Phenotype | 0.2761 | 0 |
| | Dose_binC | 0.3708 | 0 |
| CYP3A4B | Cyp3A4B_Phenotype | 0.1467 | 0 |
| | Dose_binC | 0.4429 | 0 |
| HTR2A | HTR2A_Phenotype | 0.2206 | 0 |
| | Dose_binC | 0.4174 | 0 |
| OPRM1B | OPRM1B_phenotype | 0.9065 | 0 |
| | Dose_binC | 0.4449 | 0 |
| SLC6A4 | SLC6A4_Phenotype | 0.2687 | 0 |
| | Dose_binC | 0.5245 | 0 |

TABLE 8
Percentage Non-Completers Phenotype for SUBOXONE; Higher OR indicates that
patient is more likely to drop out. In the “Sign” column, 1 indicates significant
(p-value ≤ 0.05); 0 indicates not significant (p-value > 0.05).

| | | | | | 95% | 95% | |
|---|---|---|---|---|---|---|---|
| | | | | Odds | Lower | Upper | |
| Gene | Phenotype | _Phenotype | p-value | Ratio | OR | OR | Sign. |
| ADRA2A | REDUCED | TYPICAL | 0.9638 | 1.011 | 0.635 | 1.608 | 0 |
| COMT | HIGH | INTERMEDIATE | 0.2996 | 1.321 | 0.781 | 2.234 | 0 |
| | HIGH | REDUCED | 0.3770 | 1.343 | 0.698 | 2.583 | 0 |
| | INTERMEDIATE | REDUCED | 0.9587 | 1.017 | 0.543 | 1.904 | 0 |
| CYP1A2B | EM + IM | ULTRARAPID | 0.2939 | 0.777 | 0.485 | 1.244 | 0 |
| CYP2B6 | EXTENSIVE | INTERMEDIATE | 0.1946 | 0.713 | 0.428 | 1.189 | 0 |
| | EXTENSIVE | POOR | 0.5088 | 1.373 | 0.536 | 3.513 | 0 |
| | EXTENSIVE | ULTRARAPID | 0.6270 | 0.746 | 0.229 | 2.431 | 0 |
| | INTERMEDIATE | POOR | 0.1859 | 1.925 | 0.729 | 5.083 | 0 |
| | INTERMEDIATE | ULTRARAPID | 0.9411 | 1.047 | 0.312 | 3.510 | 0 |
| | POOR | ULTRARAPID | 0.4093 | 0.544 | 0.128 | 2.313 | 0 |
| CYP2C19 | EXTENSIVE | INTERMEDIATE | 0.5922 | 0.853 | 0.477 | 1.525 | 0 |
| | EXTENSIVE | POOR | 0.3001 | 0.461 | 0.106 | 1.995 | 0 |
| | EXTENSIVE | ULTRARAPID | 0.7074 | 0.814 | 0.278 | 2.385 | 0 |
| | INTERMEDIATE | POOR | 0.4297 | 0.540 | 0.117 | 2.492 | 0 |
| | INTERMEDIATE | ULTRARAPID | 0.9358 | 0.954 | 0.302 | 3.015 | 0 |
| | POOR | ULTRARAPID | 0.5311 | 1.766 | 0.298 | 10.478 | 0 |
| CYP2C9 | EXTENSIVE | INTERMEDIATE | 0.2257 | 0.725 | 0.432 | 1.219 | 0 |
| | EXTENSIVE | POOR | 0.1175 | 0.386 | 0.117 | 1.271 | 0 |
| | INTERMEDIATE | POOR | 0.3173 | 0.533 | 0.155 | 1.831 | 0 |
| CYP2D6 | EXTENSIVE | INTERMEDIATE | 0.4422 | 0.797 | 0.446 | 1.423 | 0 |
| | EXTENSIVE | POOR | 0.1687 | 0.618 | 0.312 | 1.226 | 0 |
| | EXTENSIVE | ULTRARAPID | 0.1079 | 0.490 | 0.206 | 1.169 | 0 |
| | INTERMEDIATE | POOR | 0.5171 | 0.776 | 0.360 | 1.672 | 0 |
| | INTERMEDIATE | ULTRARAPID | 0.3130 | 0.616 | 0.240 | 1.580 | 0 |
| | POOR | ULTRARAPID | 0.6533 | 0.793 | 0.289 | 2.179 | 0 |
| CYP3A4B | EM | IM + PM | 0.1467 | 2.055 | 0.777 | 5.437 | 0 |
| HTR2A | INTERMEDIATE | NORMAL | 0.0832 | 0.569 | 0.300 | 1.077 | 0 |
| | INTERMEDIATE | REDUCED | 0.6075 | 0.871 | 0.514 | 1.475 | 0 |
| | NORMAL | REDUCED | 0.2042 | 1.531 | 0.793 | 2.957 | 0 |
| OPRM1B | INT + REDUCED | NORMAL | 0.9065 | 1.035 | 0.579 | 1.851 | 0 |
| SLC6A4 | INTERMEDIATE | NORMAL | 0.2872 | 0.750 | 0.441 | 1.274 | 0 |
| | INTERMEDIATE | REDUCED | 0.1207 | 0.614 | 0.332 | 1.137 | 0 |
| | NORMAL | REDUCED | 0.5334 | 0.819 | 0.438 | 1.533 | 0 |

Table 9 lists the p-value and significance status of phenotype and drug dose-bin-category and Table 10 lists the odds ratio and significance level for phenotypes for both Methadone and Suboxone. Dose-bin-category was not significant for any genes. Significant non-completion (%) differences were observed among phenotypes for SLC6A4, but not for any other genes. For COMT, patients with a high phenotype were 1.82 times more likely not to complete the 24-week treatment compared to patients with a reduced phenotype. For HTR2A, patients with a normal phenotype were 1.75 times more likely not to complete the treatment. For SLC6A4, patients with a reduced phenotype were 1.91 times more likely not to complete the 24-week treatment compared to those with an intermediate phenotype.

TABLE 9
Percentage Non-Completers Phenotype Overall Test for BOTH.
In the “Sign” column, 1 indicates significant (p-value ≤0.05);
0 indicates not significant (p-value >0.05).

| Gene | Factor | p-value | Sign. |
|---|---|---|---|
| ADRA2A | ADRA2A_Phenotype | 0.8021 | 0 |
| | Dose_binC | 0.2021 | 0 |
| COMT | COMT_Phenotype | 0.0910 | 0 |
| | Dose_binC | 0.2108 | 0 |
| CYP1A2B | CYP1A2B_phenotype | 0.4984 | 0 |
| | Dose_binC | 0.2083 | 0 |
| CYP2B6 | CYP2B6_Phenotype | 0.7273 | 0 |
| | Dose_binC | 0.2157 | 0 |
| CYP2C19 | CYP2C19_Phenotype | 0.8033 | 0 |
| | Dose_binC | 0.2233 | 0 |
| CYP2C9 | CYP2C9_Phenotype | 0.4960 | 0 |
| | Dose_binC | 0.2048 | 0 |
| CYP2D6 | CYP2D6_Phenotype | 0.3827 | 0 |
| | Dose_binC | 0.1814 | 0 |
| CYP3A4B | Cyp3A4B_Phenotype | 0.0847 | 0 |
| | Dose_binC | 0.1965 | 0 |
| HTR2A | HTR2A_Phenotype | 0.0792 | 0 |
| | Dose_binC | 0.1783 | 0 |
| OPRM1B | OPRM1B_phenotype | 0.6013 | 0 |
| | Dose_binC | 0.1995 | 0 |
| SLC6A4 | SLC6A4_Phenotype | 0.0272 | 1 |
| | Dose_binC | 0.2261 | 0 |

TABLE 10
Percentage Non-Completers Phenotype for BOTH; Higher Odds Ratio (OR) indicates
that patient is more likely to drop out. In the “Sign” column, 1 indicates significant
(p-value ≤ 0.05); 0 indicates not significant (p-value > 0.05).

| | | | | | 95% | 95% | |
|---|---|---|---|---|---|---|---|
| | | | | Odds | Lower | Upper | |
| Gene | Phenotype | _Phenotype | p-value | Ratio | OR | OR | Sign. |
| ADRA2A | REDUCED | TYPICAL | 0.8021 | 1.049 | 0.724 | 1.519 | 0 |
| COMT | HIGH | INTERMEDIATE | 0.1915 | 1.320 | 0.870 | 2.003 | 0 |
| | HIGH | REDUCED | 0.0312 | 1.823 | 1.056 | 3.147 | 1 |
| | INTERMEDIATE | REDUCED | 0.2228 | 1.381 | 0.822 | 2.320 | 0 |
| CYP1A2B | EM + IM | ULTRARAPID | 0.4984 | 0.879 | 0.605 | 1.277 | 0 |
| CYP2B6 | EXTENSIVE | INTERMEDIATE | 0.6925 | 0.921 | 0.611 | 1.387 | 0 |
| | EXTENSIVE | POOR | 0.7426 | 0.892 | 0.449 | 1.769 | 0 |
| | EXTENSIVE | ULTRARAPID | 0.2664 | 0.601 | 0.245 | 1.475 | 0 |
| | INTERMEDIATE | POOR | 0.9292 | 0.968 | 0.476 | 1.968 | 0 |
| | INTERMEDIATE | ULTRARAPID | 0.3637 | 0.653 | 0.260 | 1.638 | 0 |
| | POOR | ULTRARAPID | 0.4716 | 0.674 | 0.231 | 1.971 | 0 |
| CYP2C19 | EXTENSIVE | INTERMEDIATE | 0.9211 | 0.977 | 0.616 | 1.550 | 0 |
| | EXTENSIVE | POOR | 0.3208 | 0.585 | 0.203 | 1.687 | 0 |
| | EXTENSIVE | ULTRARAPID | 0.9811 | 1.011 | 0.403 | 2.538 | 0 |
| | INTERMEDIATE | POOR | 0.3656 | 0.598 | 0.197 | 1.820 | 0 |
| | INTERMEDIATE | ULTRARAPID | 0.9451 | 1.035 | 0.389 | 2.756 | 0 |
| | POOR | ULTRARAPID | 0.4318 | 1.730 | 0.441 | 6.782 | 0 |
| CYP2C9 | EXTENSIVE | INTERMEDIATE | 0.7709 | 0.940 | 0.618 | 1.428 | 0 |
| | EXTENSIVE | POOR | 0.2393 | 0.582 | 0.237 | 1.433 | 0 |
| | INTERMEDIATE | POOR | 0.3186 | 0.620 | 0.242 | 1.587 | 0 |
| CYP2D6 | EXTENSIVE | INTERMEDIATE | 0.9745 | 0.992 | 0.624 | 1.579 | 0 |
| | EXTENSIVE | POOR | 0.2102 | 0.713 | 0.420 | 1.211 | 0 |
| | EXTENSIVE | ULTRARAPID | 0.1884 | 0.624 | 0.309 | 1.260 | 0 |
| | INTERMEDIATE | POOR | 0.2833 | 0.718 | 0.392 | 1.315 | 0 |
| | INTERMEDIATE | ULTRARAPID | 0.2324 | 0.628 | 0.293 | 1.347 | 0 |
| | POOR | ULTRARAPID | 0.7452 | 0.875 | 0.392 | 1.955 | 0 |
| CYP3A4B | EM | IM + PM | 0.0847 | 1.954 | 0.913 | 4.185 | 0 |
| HTR2A | INTERMEDIATE | NORMAL | 0.0285 | 0.573 | 0.348 | 0.943 | 1 |
| | INTERMEDIATE | REDUCED | 0.1962 | 0.758 | 0.498 | 1.154 | 0 |
| | NORMAL | REDUCED | 0.2908 | 1.324 | 0.787 | 2.229 | 0 |
| OPRM1B | INT + REDUCED | NORMAL | 0.6013 | 1.130 | 0.714 | 1.789 | 0 |
| SLC6A4 | INTERMEDIATE | NORMAL | 0.1684 | 0.740 | 0.483 | 1.136 | 0 |
| | INTERMEDIATE | REDUCED | 0.0076 | 0.521 | 0.323 | 0.841 | 1 |
| | NORMAL | REDUCED | 0.1610 | 0.704 | 0.431 | 1.150 | 0 |

### Combinatorial Gene Modeling

We next undertook a combinatorial approach to assess whether there was a composite genetic risk score having a significant association with completion rate. Risk scores were assigned using two different models, a “biology-based” model and a “data-based” model. The biology based risk scores were assigned using the most biologically appropriate hypothesis for a given genotype or haplotype, for example, based upon the enzyme activity reported to be associated with that genotype or haplotype in the scientific literature (e.g., as shown in Table 1 above) in combination with current biological understanding of the drug including both its pharmacokinetics (how it is absorbed, distributed, metabolized and excreted) and its pharmacodynamics (how it effects the body).

In contrast, the data-based risk scores were assigned based on how the phenotype of the genotype or haplotype for each gene was associated with non-completion in the single-gene analysis above. For example, a patient may be scored 0 if his/her phenotype is reduced, 0.5 if intermediate, and 1 if high. Tables 11 and 12 show how the risk scores were assigned in each of these models.

TABLE 11
Scoring scheme for Data Based Model

| | PHENOTYPE | | | |
|---|---|---|---|---|
| | Val/Val | Val/Met | Met/Met | |
| COMT | 0 | 0.5 | 1 | |
| SLC6A4 | S/S | L/S | L/L | |
| | 1 | 0 | 0 | |
| HTR2A | C/C | C/T | T/T | |
| | 0.5 | 0 | 1 | |
| CYP3A4 | PM | IM | EM | |
| | 0 | 0 | 1 | |
| | PM | IM | EM | UM |
| CYP2B6 | 1 | 0 | 0 | 1 |
| CYP2C19 | 1 | 0 | 0 | 1 |
| CYP1A2 | 1 | 0 | 0 | 1 |
| CYP2D6 | 1 | 0 | 0 | 1 |
| CYP2C9 | 1 | 0 | 0 | 1 |

TABLE 12
Scoring scheme for Biology Based Model

| | PHENOTYPE | | | |
|---|---|---|---|---|
| | Val/Val | Val/Met | Met/Met | |
| COMT | 0 | 0.5 | 1 | |
| SLC6A4 | S/S | L/S | L/L | |
| | 1 | 0.5 | 0 | |
| HTR2A | C/C | C/T | T/T | |
| | 0 | 0.5 | 1 | |
| CYP3A4 | PM | IM | EM | |
| | 0 | 0 | 1 | |
| | Poor | IM | EM | UM |
| CYP2B6 | 0 | 0 | 1 | 1 |
| CYP2C19 | 0 | 0 | 1 | 1 |
| CYP1A2 | 0 | 0 | 1 | 1 |
| CYP2D6 | 0 | 0 | 1 | 1 |
| CYP2C9 | 0 | 0 | 1 | 1 |

We then used a composite risk score, which is the sum of the individual risk scores for each gene of interest, to assess the impact of various combinations of genetic markers on the ability of the patients to complete the 24-week drug treatment regimen. The completion/non-completion was modeled using a logistic regression which included the total genetic risk score and dose-bin-category. This model assesses the effect of the total genetic risk score on the non-completion rate while adjusting for drug dose level. The likelihood of non-completion with respect to the total genetic risk score is represented by the odds ratio. If a p-value is <0.05, then it is considered that statistically significant that the non-completion is associated with the total genetic risk score.

Using this approach, we identified additional genes and combinations of genes having a significant association with completers or non-completers. In particular, we found that the following combinations of genetic markers had a statistically significant effect on 24-week treatment completion for MET and BUP: COMT-CYP3A4, COMT-HTR2A, COMT-SLC6A4, COMT-HTR2A-CYP3A4, COMT-SLC6A4-CYP3A4, COMT-SLC6A4-HTR2A, SLC6A4-HTR2A-CYP3A4, COMT-SLC6A4-HTR2A-CYP3A4 (4G), 4G-CYP2B6, 4G-CYP2C19, 4G-CYP1A2, 4G-CYP2C9, 4G-CYP2D6, 4G-CYP2B6-CYP2C19, 4G-CYP2B6-CYP1A2, 4G-CYP2B6-2C9, 4G-CYP2B6-CYP2D6, 4G-CYP2C19-CYP1A2, 4G-CYP2C19-CYP2C9, 4G-CYP2C19-CYP2D6, 4G-CYP2B6-CYP2C9-CYP2D6, 4G-CYP2B6-CYP2C9-CYP2C19-CYP2D6. The higher the risk score, the more likely the patients did not complete the treatment program. Generally the likelihood of patients not completing the treatment program became more significant with the number of genes (2 to 9).

In the methadone (MET) arm, using the biology-based model, 7 of 25 composite endpoints were significant when adjusted for Dose-bin. In the buprenorphine (BUP) arm, none of the 25 composite endpoints were significant when adjusted for Dose-bin. When the two arms were combined, 11 of 25 composite endpoints were significant.

Using the data-based model, 23 of 25 composite endpoints for the MET arm were significant when adjusted for Dose-bin. In the BUP arm, 21 of 25 composite endpoints were significant when adjusted for Dose-bin. When the two arms were combined, 25 of 25 composite endpoints were significant.

TABLE 13
Results for Biology and Data Based Models Showing Combinations of Genetic
Variants Having a Significant Effect on Outcome. The symbol “•” denotes statistical
significance at 0.05 level of significance.

| | | Biology-Based | | | Data-Based | | |
|---|---|---|---|---|---|---|---|
| | Description | MET | BUP | BOTH | MET | BUP | BOTH |
| 1 | COMT-CYP3A4 | • | | • | • | | • |
| 2 | COMT-HTR2A | | | • | • | | • |
| 3 | COMT-SLC6A4 | | | • | | | • |
| 4 | HTR2A-CYP3A4 | | | | • | | • |
| 5 | SLC6A4-HTR2A | | | | | • | • |
| 6 | SLC6A4-CYP3A4 | | | | • | • | • |
| 7 | COMT-HTR2A-CYP3A4 | | | | • | • | • |
| 8 | COMT-SLC6A4-CYP3A4 | • | | • | • | • | • |
| 9 | COMT-SLC6A4-HTR2A | | | • | • | • | • |
| 10 | SLC6A4-HTR2A-CYP3A4 | • | | • | • | • | • |
| 11 | COMT-SLC6A4-HTR2A- | • | | • | • | • | • |
| | CYP3A4 (ORG4) | | | | | | |
| 12 | ORG4-CYP2B6 | | | | • | • | • |
| 13 | ORG4-CYP2C19 | | | • | • | • | • |
| 14 | ORG4-CYP2B6-CYP2C19 | | | | • | • | • |
| 15 | ORG4-CYP1A2 | • | | • | • | • | • |
| 16 | ORG4-CYP2C9 | • | | | • | • | • |
| 17 | ORG4-CYP2D6 | | | | • | • | • |
| 18 | ORG4-CYP2B6-1A2 | | | • | • | • | • |
| 19 | ORG4-CYP2B6-2C9 | | | | • | • | • |
| 20 | ORG4-CYP2B6-2D6 | | | | • | • | • |
| 21 | ORG4-CYP2C19-1A2 | | | • | • | • | • |
| 22 | ORG4-CYP2C19-2C9 | • | | | • | • | • |
| 23 | ORG4-CYP2C19-2D6 | | | | • | • | • |
| 24 | ORG4-CYP2B6-2C9-2D6 | | | | • | • | • |
| 25 | ALL9 | | | | • | • | • |

TABLE 14
Odds Ratio Modeling for Non-Completers: the higher the odds ratio, more likely to drop

| | Biology-Based | | | Data-Based | | |
|---|---|---|---|---|---|---|
| Model | SUBOXONE | METHADONE | BOTH | SUBOXONE | METHADONE | BOTH |
| COMT-CYP3A4 | 1.6 | 1.98 | 1.78 | 1.6 | 1.98 | 1.78 |
| COMT-HTR2A | 1.34 | 1.87 | 1.47 | 1.51 | 2.44 | 1.81 |
| COMT-SLC6A4 | 1.27 | 2.12 | 1.55 | 1.46 | 2.24 | 1.74 |
| HTR2A-CYP3A4 | 1.53 | 1.31 | 1.4 | 1.73 | 1.73 | 1.77 |
| SLC6A4-HTR2A | 1.21 | 1.5 | 1.25 | 1.54 | 2 | 1.71 |
| SLC6A4-CYP3A4 | 1.31 | 1.72 | 1.45 | 1.54 | 1.97 | 1.7 |
| COMT-HTR2A-CYP3A4 | 1.44 | 1.71 | 1.52 | 1.55 | 2.11 | 1.76 |
| COMT-SLC6A4-CYP3A4 | 1.37 | 1.91 | 1.57 | 1.52 | 2.02 | 1.71 |
| COMT-SLC6A4-HTR2A | 1.27 | 1.84 | 1.42 | 1.49 | 2.27 | 1.75 |
| SLC6A4-HTR2A-CYP3A4 | 1.32 | 1.51 | 1.35 | 1.56 | 1.92 | 1.7 |
| COMT-SLC6A4-HTR2A-CYP3A4 | 1.33 | 1.74 | 1.46 | 1.51 | 2.1 | 1.72 |
| ORG4-CYP2B6 | 1.14 | 1.44 | 1.25 | 1.37 | 2.29 | 1.66 |
| ORG4-CYP2C19 | 1.16 | 1.53 | 1.27 | 1.48 | 1.76 | 1.6 |
| ORG4-CYP2B6-CYP2C19 | 1.06 | 1.38 | 1.17 | 1.36 | 1.9 | 1.56 |
| ORG4-CYP1A2 | 1.36 | 1.74 | 1.47 | 1.4 | 1.56 | 1.47 |
| ORG4-CYP2C9 | 1.09 | 1.56 | 1.24 | 1.54 | 2.07 | 1.72 |
| ORG4-CYP2D6 | 1.14 | 1.41 | 1.24 | 1.55 | 1.93 | 1.69 |
| ORG4-CYP2B6-1A2 | 1.15 | 1.44 | 1.26 | 1.32 | 1.69 | 1.45 |
| ORG4-CYP2B6-2C9 | 1.01 | 1.4 | 1.16 | 1.4 | 2.24 | 1.66 |
| ORG4-CYP2B6-2D6 | 1.05 | 1.28 | 1.15 | 1.45 | 2.12 | 1.67 |
| ORG4-CYP2C19-1A2 | 1.17 | 1.53 | 1.28 | 1.4 | 1.46 | 1.44 |
| ORG4-CYP2C19-2C9 | 1.02 | 1.44 | 1.16 | 1.52 | 1.77 | 1.62 |
| ORG4-CYP2C19-2D6 | 1.06 | 1.34 | 1.16 | 1.53 | 1.72 | 1.61 |
| ORG4-CYP2B6-2C9-2D6 | 0.97 | 1.3 | 1.1 | 1.47 | 2.08 | 1.67 |
| ALL9 | 0.95 | 1.27 | 1.07 | 1.4 | 1.58 | 1.47 |

The current study suggests that functional polymorphisms related to synaptic dopamine or serotonin levels may predict non-completion during methadone treatment. In particular, patients with the S/S genotype at 5-HTTLPR in SLC6A4 or the Val/Val genotype Val158Met (rs4680) in COMT may require additional treatment, either pharmaceutical or psychiatric, to improve their chances of completing treatment for their addiction. Dose level played an important role in likelihood of completion in Methadone treatment. The total genetic risk score methods proved to be a good way to evaluate the potential combinatorial effect of genes on non-completion of Methadone and Suboxone™ treatment. Generally, the higher the risk score, the more likely patients were to not complete the treatment.

### Example 2

The aim of this study was to perform exploratory modeling of pharmacodynamic (‘PD’), pharmacokinetic (‘PK’), and mixed PK/PD models to demonstrate the general applicability of using models incorporating the genes and panels of the present disclosure to distinguish completers from non-completers to Methadone treatment.

### Model Evaluations

For the model analyses, models were developed that sort patients into one of three categories, Green (‘G’) Yellow (‘Y’) or Red (‘R’). Patients determined to be high risk for non-completion by the model are placed into the Red Bin. Patients at intermediate risk of non-completion are placed in the Yellow Bin, and for this study, Yellow Bin patients are treated the same as patients found to be at Low Risk of non-completion, or those who are placed in the Green Bin. Thus, the Green and Yellow bins are combined for analysis.

### Model Descriptions

The GeneSight Analgesic test (commercial test available from Assurex Health, Inc.) was used as a baseline to determine High Risk patients entering methadone maintenance therapy. Furthremore, the following PK, PD and PK/PD models were evaluated.

The PK model represents a physiologically based pharmacokinetic algorithm that combines combinatorial pharmacogenetics with a physiological algorithm for predicting medication exposure, as described elsewhere herein. Model assignments to G Y or R were based on the therapeutic ranges provided by the literature for methadone as shown in Table 15.

TABLE 15
PK Model Assignments Based on Predicted Exposure

| PK Predicted Exposure Range (ng/mL) | Bin |
|---|---|
| >=300 | Green |
| <300 and >=200 | Yellow |
| <200 | Red |

Three separate PD models were evaluated, based on the genotypes in Table 1.3:

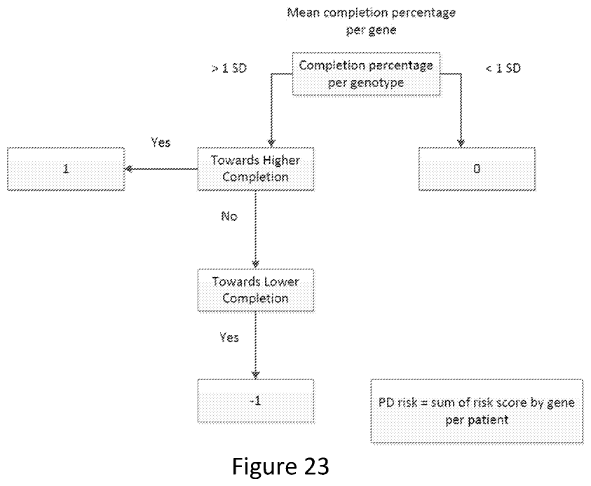

PD Model 1: The completion percentage was calculated for each genotype. For each gene and genotype completion percentage, the means and standard deviations were calculated. For each genotype that was greater than or equal to one standard deviation away from the mean, towards higher completion, a score of 1 was given. For each genotype that was greater than or equal to one standard deviation away from the mean, towards lower completion, a score of −1 was given. All others received a score of 0. See FIG. 23. The patient's PD risk was calculated by summing the total genotype risk score. The individual risk scores were combined to create the SumRisk variable; a final score of −1 was converted to Green, a score of 0 was converted to Yellow, and a score of 1 was converted to Red. FIG. 23 shows a flow chart of PD Model 1. A listing of tested alleles, and score assignments for PD Model 1 can be found in Table 16.

TABLE 16
Genotype Score Assignements for PD Model 1

| ADRA2A | | COMT | | OPRM1 | | HTR2A | | SLC6A4 | |
|---|---|---|---|---|---|---|---|---|---|
| Genotype | Score | Genotype | Score | Genotype | Score | Genotype | Score | Genotype | Score |
| CC | 0 | MM | −1 | AA | 0 | AA | 0 | LL | 0 |
| CG | 0 | VM | 0 | AG | 0 | GA | 0 | LS | 0 |
| GG | 0 | VV | 0 | GG | 0 | GG | 0 | SS | 1 |

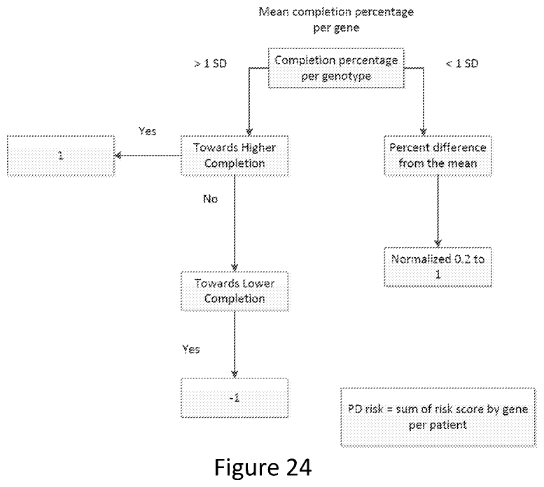

PD Model 2: The completion percentage was calculated for each genotype. For each gene and genotype completion percentage, the means and standard deviations were calculated. For each genotype that was one standard deviation away from the mean, towards higher completion, a score of 1 was given. For each genotype that was one standard deviation away from the mean, towards lower completion, a score of −1 was given. For genotypes that were not one standard deviation away, the raw percent difference from the mean was observed as the score. Each score was then normalized across each gene using a scale from 0.2 to 1. See FIG. 24. Each value was normalized across all genes by converting the percentages on a plus/minus scale of 0, 0.2, 0.5, and 1 according to Table 17. Values that were greater than or equal to 1 SD away were automatically scored as a 1 or −1. The conversion of genotypes to scores was as shown in Tabl 18. The patient's PD risk was calculated by summing the total genotype risk score. Bins were assigned over the range of scores that maximized completers in Green and Yellow bins and non-completers in the Red bin.

TABLE 17
PD Score Conversions Model 2

| Δ % from mean completion | Normalized Score |
|---|---|
| 0 | 0 |
| 0.1-0.3 | −0.2 or 0.2 |
| 0.4-0.7 | −0.5 or 0.5 |
| 0.8-1 or greater | −1 or 1 |

TABLE 18
Genotype Score Conversions for PD Model 2

| | | |
|---|---|---|
| Genotype | Score | Conversion |

ADRA2A

| | | |
|---|---|---|
| CC | 0.3 | 0.2 |
| CG | 0.1 | 0.2 |
| GG | −1 | −1 |

COMT

| | | |
|---|---|---|
| MM | −1.1 | −1 |
| VM | 0.4 | .5 |
| VV | 0.4 | .5 |

OPRM1

| | | |
|---|---|---|
| AA | 1 | 1 |
| AG | −1 | −1 |
| GG | 0 | 0 |

HTR2A

| | | |
|---|---|---|
| AA | −0.1 | −0.2 |
| GA | −1 | −1 |
| GG | 1 | 1 |

SLC6A4

| | | |
|---|---|---|
| LL | −0.3 | −0.2 |
| LS | −0.4 | −0.5 |
| SS | 1.1 | 1 |

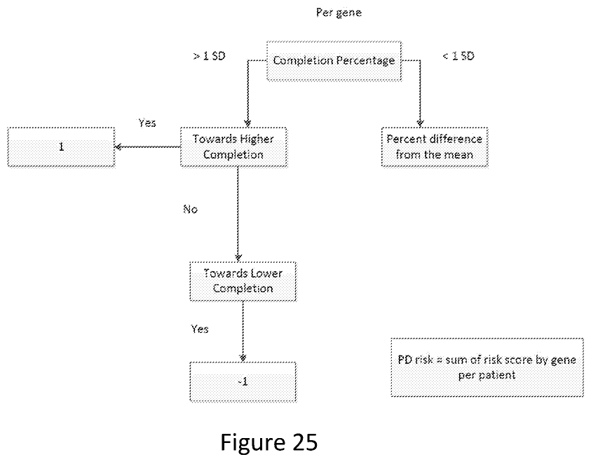

PD Model 3: The completion percentage was calculated for each genotype. For each genotype, the raw percent difference of completion compared to the mean of completion was assigned as the genotype's PD score. See FIG. 25. The genotypes were assigned scores as shown in Table 19. The patient's PD risk was calculated by summing the total genotype risk score. Bins were assigned over the range of scores that maximized completers in Green and Yellow bins and non-completers in the Red bin.

TABLE 19
Genotype Score Assignments for PD Model 3

| ADRA2A | | COMT | | OPRM1 | | HTR2A | | SLC6A4 | |
|---|---|---|---|---|---|---|---|---|---|
| Genotype | Score | Genotype | Score | Genotype | Score | Genotype | Score | Genotype | Score |
| CC | 0.3 | MM | −0.8 | AA | 0.1 | AA | −0.1 | LL | −0.3 |
| CG | 0.1 | VM | 0.4 | AG | −0.1 | GA | −0.6 | LS | −0.4 |
| GG | −0.5 | VV | 0.4 | GG | 0 | GG | 0.6 | SS | 0.7 |

Three separate PK/PD Models were generated by combining the PK model with each of the three PD models separately as follows. For each PKPD model, the corresponding PD risk score was added to the raw PK score (bin). A total PD risk score that was negative, represents a reduction of the baseline PK score, while a total PD risk score that was positive represented an increase in the baseline PK score.

### Methods

Samples from patients treated with Methadone in the START trial described in Example 1 above were used (N=352). The samples were randomized into two samples, Sample 1 (N=176; 156 completers and 20 non-completers) and Sample 2 (N=176; 159 completers and 17 non-completers). Each model was tested as described below against Sample 1, Sample 2 and/or the Full Sample.

Pearson's product moment, Chi-square with Yates correction were calculated. Diagnostic tests, odds ratios, Pearson's product moment, Chi-square with Yates correction were calculated using MEDCALC and R version 3.3.1.

### Results

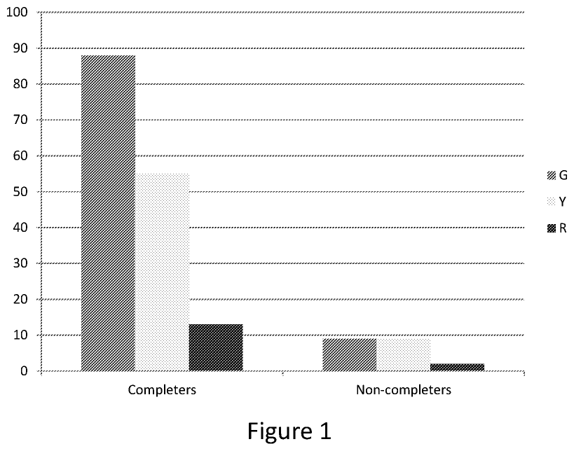

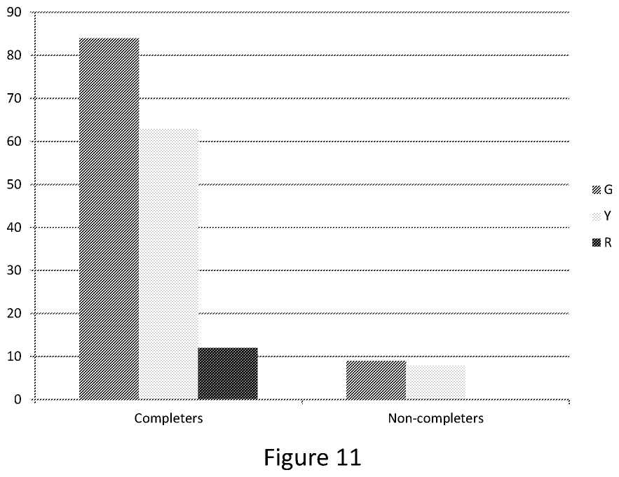

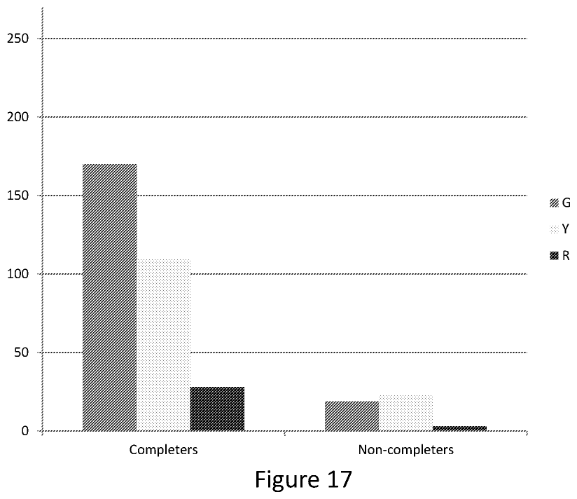

The Results for the baseline comparator (GeneSight Analgesic test) are shown in FIGS. 1, 11 and 17 and Tables 20-25. FIG. 1 and Tables 20-21 show the results for the baseline comparator for Sample 1. FIG. 11 and Tables 22-23 show the results for the baseline comparator for Sample 2. FIG. 17 and Tables 24-25 show the results for the baseline comparator for the Full Sample.

TABLE 20
Binning Results for Baseline Model, Sample 1

| Bin | Completers | Non Completers |
|---|---|---|
| Green | 88 | 9 |
| Yellow | 55 | 9 |
| Red | 13 | 2 |

Fisher's Exact Test P = 0.6177

TABLE 21
Baseline Model Evaluation, Sample 1

| | Risk | | | |
|---|---|---|---|---|
| | Non | | | |
| Test Result | Completer | N | Completer | N |
| Positive (Red) | True Positive | 2 | False Positive | 13 |
| Negative | False Negative | 18 | True Negative | 143 |
| (Green or | | | Disease | |
| Yellow) | Evaluation | % | Prevalence (%) | |
| | Sensitivity | 10.00 | 11.36 | |
| | Specificity | 91.67 | | |

TABLE 22
Binning Results for Baseline Model, Sample 2

| Bin | Completers | Non Completers |
|---|---|---|
| Green | 84 | 9 |
| Yellow | 63 | 8 |
| Red | 12 | 0 |

Fisher's Exact Test P = 0.4747

TABLE 23
Baseline Model Evaluation, Sample 2

| | Risk | | | |
|---|---|---|---|---|
| | Non | | | |
| Test Result | Completer | N | Completer | N |
| Positive (Red) | True Positive | 0 | False Positive | 12 |
| Negative | False Negative | 17 | True Negative | 147 |
| (Green or | | | Disease | |
| Yellow) | Evaluation | % | Prevalence (%) | |
| | Sensitivity | 0.00 | 9.66 | |
| | Specificity | 92.45 | | |

TABLE 24
Binning Results for Baseline Model, Full Sample

| Bin | Completers | Non Completers |
|---|---|---|
| Green | 170 | 19 |
| Yellow | 109 | 23 |
| Red | 28 | 3 |

Fisher's Exact Test P = 0.1393

TABLE 25
Baseline Model Evaluation, Full Sample

| | Risk | | | |
|---|---|---|---|---|
| | Non | | | |
| Test Result | Completer | N | Completer | N |
| Positive (Red) | True Positive | 3 | False Positive | 28 |
| Negative | False Negative | 42 | True Negative | 279 |
| (Green or | | | Disease | |
| Yellow) | Evaluation | % | Prevalence (%) | |
| | Sensitivity | 6.67 | 12.78 | |
| | Specificity | 90.88 | | |

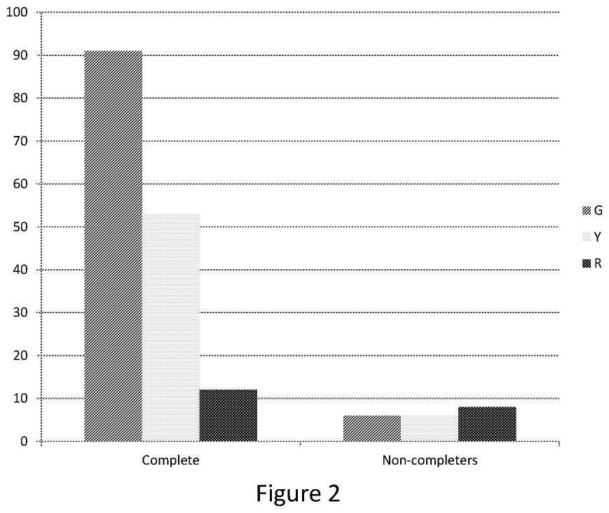

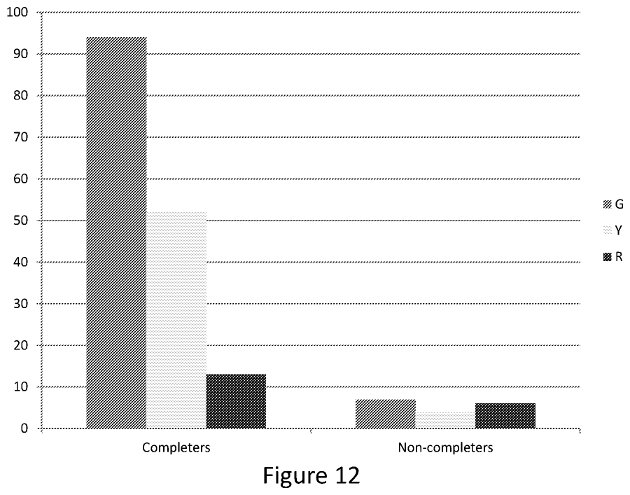

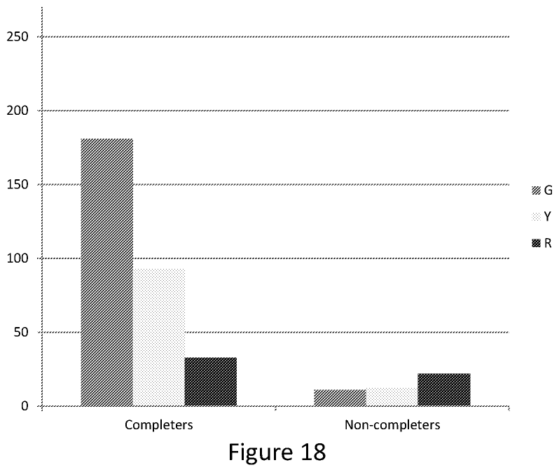

The results for the PK model are shown in FIGS. 2, 12, and 18, and Tables 26-31. FIG. 2 and Tables 26-27 show the results for the PK model for Sample 1. FIG. 12 and Tables 28-29 show the results for the PK model for Sample 2. FIG. 18 and Tables 30-31 show the results for the PK model for the Full Sample.

TABLE 26
Binning Results for PK Model, Sample 1

| Bin | Completers | Non Completers |
|---|---|---|
| Green | 91 | 6 |
| Yellow | 53 | 6 |
| Red | 12 | 8 |

Chi-squared test X = 18.949, P = 7.68 × 10 − 5

TABLE 27
PK Model Evaluation, Sample 1

| | Risk | | | |
|---|---|---|---|---|
| | Non | | | |
| Test Result | Completer | N | Completer | N |
| Positive (Red) | True Positive | 8 | False Positive | 12 |
| Negative | False Negative | 12 | True Negative | 144 |
| (Green or | | | Disease | |
| Yellow) | Evaluation | % | Prevalence (%) | |
| | Sensitivity | 40.00 | 11.36 | |
| | Specificity | 92.31 | | |

TABLE 28
Binning Results for PK Model, Sample 2

| Bin | Completers | Non Completers |
|---|---|---|
| Green | 94 | 7 |
| Yellow | 52 | 4 |
| Red | 13 | 6 |

TABLE 29
PK Model Evaluation, Sample 2

| | Risk | | | |
|---|---|---|---|---|
| | Non | | | |
| Test Result | Completer | N | Completer | N |
| Positive (Red) | True Positive | 6 | False Positive | 13 |
| Negative | False Negative | 11 | True Negative | 146 |
| (Green or | | | Disease | |
| Yellow) | Evaluation | % | Prevalence (%) | |
| | Sensitivity | 35.29 | 9.66 | |
| | Specificity | 91.82 | | |

TABLE 30
Binning Results for PK Model, Full Sample

| Bin | Completers | Non Completers |
|---|---|---|
| Green | 181 | 11 |
| Yellow | 93 | 12 |
| Red | 33 | 22 |

Chi-squared X = 45.282 P = 1.47 × 10 − 10

TABLE 31
PK Model Evaluation, Full Sample

| | Risk | | | |
|---|---|---|---|---|
| | Non | | | |
| Test Result | Completer | N | Completer | N |
| Positive (Red) | True Positive | 22 | False Positive | 33 |
| Negative | False Negative | 23 | True Negative | 274 |
| (Green or | | | | |
| Yellow) | | | | |
| | | | Disease | |
| | Evaluation | % | Prevalence (%) | |
| | Sensitivity | 48.89 | 12.78 | |
| | Specificity | 89.25 | | |

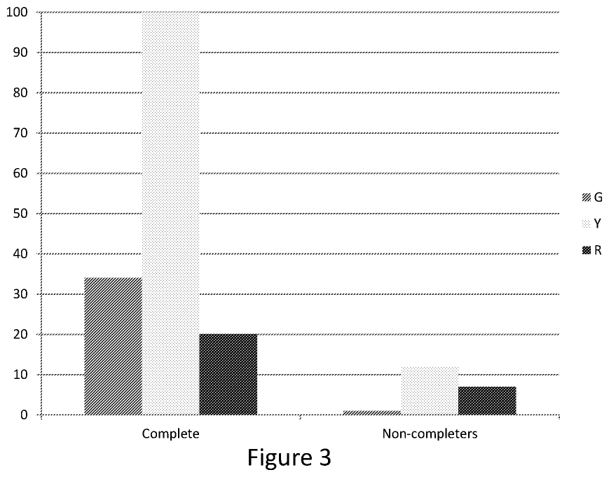

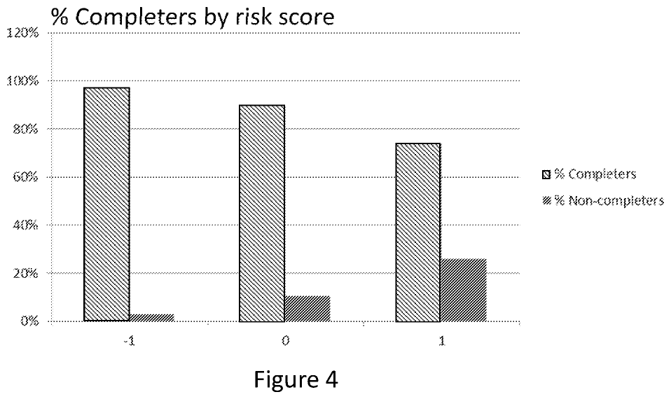

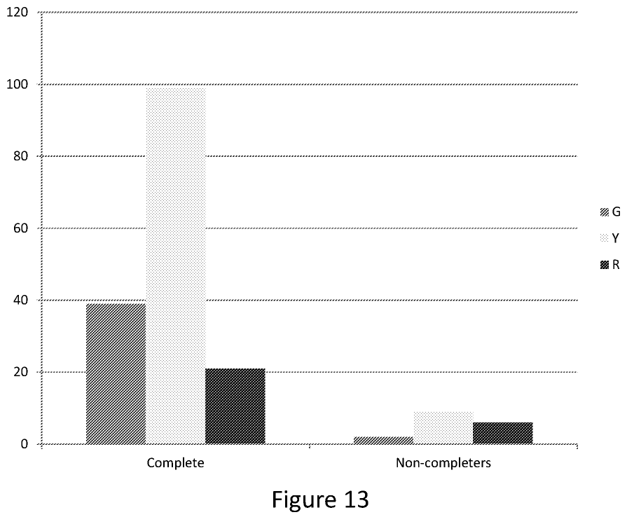

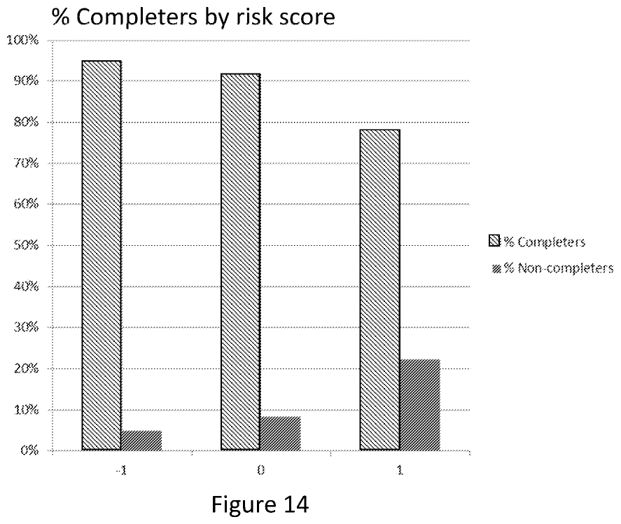

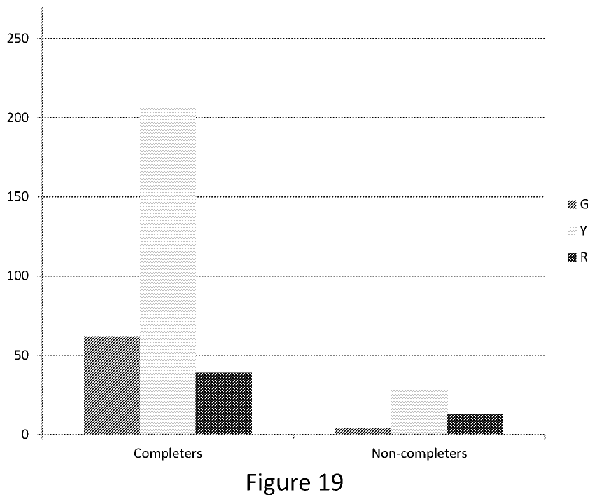

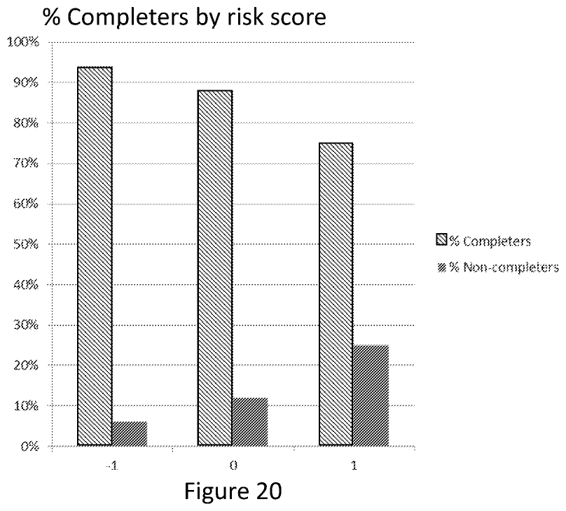

The results for the PD Model 1 are shown in FIGS. 3, 4, 13, 14, 19, and 20 and Tables 32-46. FIGS. 3 and 4 and Tables 32-46 show the results for PD Model 1 for Sample 1. FIGS. 13 and 14 and Tables 37-41 show the results for PD Model 1 for Sample 2. FIGS. 19 and 20 and Tables 42-46 show the results for PD Model 1 for the Full Sample.

TABLE 32
Univariate and Multivariate Analysis of PD 1 genes, Sample 1

| PD Model 1 | Test | Significance | Correlation |
|---|---|---|---|
| COMT Met/Met = Low | (completers, | P = 0.03179 | r = −0.16 |
| Risk (−1) | riskCOMT) | | |
| SLC6A4 S/S = High Risk | (completers, | P = 0.07279 | r = −0.14 |
| (1) | riskSLC6A4) | | |
| COMTrisk + SLC6A4risk = | (completers, | P = 0.00537 | r = −0.21 |
| SumRisk | SumRisk) | | |

TABLE 33
COMT and SLC6A4 Results for PD Model 1, Sample 1

| COMT | SLC6A4 | Completers | Non Completers | Bin |
|---|---|---|---|---|
| Normal Risk | Normal Risk | 94 | 12 | Yellow |
| Low Risk | Normal Risk | 34 | 1 | Green |
| Low Risk | High Risk | 8 | 0 | Yellow |
| Normal Risk | High Risk | 20 | 7 | Red |

TABLE 34
Odds Ratios for non-completion for PD Model 1, Sample 1

| Combination Risk Phenotypes | | |
|---|---|---|
| Exposed vs. Control | Odds Ratio | Significance |
| NR/NR vs. NR/NR | Reference | Reference |
| LR/NR vs. NR/NR | 0.2304 | P = 0.1660 |
| LR/HR vs. NR/NR | 0.4447 | P = 0.5855 |
| NR/HR vs. NR/NR | 2.7417 | P = 0.0597 |
| LR/NR vs. NR/HR | 0.0840 | P = 0.0251 |

TABLE 35
Binning Results for PD Model 1, Sample 1

| Bin | Completers | Non Completers |
|---|---|---|
| Green | 34 | 1 |
| Yellow | 102 | 12 |
| Red | 20 | 7 |

TABLE 36
PD Model 1 Evaluation, Sample 1

| | Risk | | | |
|---|---|---|---|---|
| | Non | | | |
| Test Result | Completer | N | Completer | N |
| Positive (Red) | True Positive | 7 | False Positive | 20 |
| Negative | False Negative | 13 | True Negative | 136 |
| (Green or | | | | |
| Yellow) | | | | |
| | | | Disease | |
| | Evaluation | % | Prevalence (%) | |
| | Sensitivity | 35.00 | 11.36 | |
| | Specificity | 87.18 | | |

TABLE 37
Univariate and Multivariate Analysis of PD 1 genes, Sample 2

| PD Model 1 | Test | Significance | Correlation |
|---|---|---|---|
| COMT Met/Met = Low | (completers, | P = 0.1324 | r = −0.11 |
| Risk (−1) | riskCOMT) | | |
| SLC6A4 S/S = High Risk | (completers, | P = 0.08 | r = −0.13 |
| (1) | riskSLC6A4) | | |
| COMTrisk + SLC6A4risk = | (completers, | P = 0.02674 | r = −0.17 |
| SumRisk | SumRisk) | | |

TABLE 38
COMT and SLC6A4 Results for PD Model 1, Sample 2

| COMT | SLC6A4 | Completers | Non Completers | Bin |
|---|---|---|---|---|
| Normal Risk | Normal Risk | 92 | 9 | Yellow |
| Low Risk | Normal Risk | 39 | 2 | Green |
| Low Risk | High Risk | 7 | 0 | Yellow |
| Normal Risk | High Risk | 21 | 6 | Red |

TABLE 39
Odds Ratios for non-completion for PD Model 1, Sample 2

| Combination Risk Phenotypes | | |
|---|---|---|
| Exposed vs. Control | Odds Ratio | Significance |
| NR/NR vs. NR/NR | Reference | Reference |
| LR/NR vs. NR/NR | 0.5242 | P = 0.4222 |
| LR/HR vs. NR/NR | 0.6491 | P = 0.7732 |
| NR/HR vs. NR/NR | 2.9206 | P = 0.0646 |
| LR/NR vs. NR/HR | 0.1795 | P = 0.0458 |

TABLE 40
Binning Results for PD Model 1, Sample 2

| Bin | Completers | Non Completers |
|---|---|---|
| Green | 39 | 2 |
| Yellow | 99 | 9 |
| Red | 21 | 6 |

TABLE 41
PD Model 1 Evaluation. Sample 2

| | Risk | | | |
|---|---|---|---|---|
| | Non | | | |
| Test Result | Completer | N | Completer | N |
| Positive (Red) | True Positive | 6 | False Positive | 21 |
| Negative | False Negative | 11 | True Negative | 138 |
| (Green or | | | | |
| Yellow) | | | | |
| | | | Disease | |
| | Evaluation | % | Prevalence (%) | |
| | Sensitivity | 35.29 | 9.66 | |
| | Specificity | 86.79 | | |

TABLE 42
Univariate and Multivariate Analysis of PD 1 genes, Full Sample

| PD Model 1 | Test | Significance | Correlation |
|---|---|---|---|
| COMT Met/Met = Low | (completers, | P = 0.04664 | r = −0.11 |
| Risk (−1) | riskCOMT) | | |
| SLC6A4 S/S = High Risk | (completers, | P = 0.0229 | r = −0.12 |
| (1) | riskSLC6A4) | | |
| COMTrisk + SLC6A4risk = | (completers, | P = 0.0028 | r = −0.16 |
| SumRisk | SumRisk) | | |

TABLE 43
COMT and SLC6A4 Results for PD Model 1, Full Sample

| COMT | SLC6A4 | Completers | Non Completers | Bin |
|---|---|---|---|---|
| Normal Risk | Normal Risk | 193 | 27 | Yellow |
| Low Risk | Normal Risk | 62 | 4 | Green |
| Low Risk | High Risk | 13 | 1 | Yellow |
| Normal Risk | High Risk | 39 | 13 | Red |

TABLE 44
Odds Ratios for non-completion for PD Model 1, Full Sample

| Combination Risk Phenotypes | | |
|---|---|---|
| Exposed vs. Control | Odds Ratio | Significance |
| NR/NR vs. NR/NR | Reference | Reference |
| LR/NR vs. NR/NR | 0.4612 | P = 0.1634 |
| LR/HR vs. NR/NR | 0.5499 | P = 0.5718 |
| NR/HR vs. NR/NR | 2.3827 | P = 0.0225 |
| LR/NR vs. NR/HR | 0.1935 | P = 0.0068 |

TABLE 45
Binning Results for PD Model 1, Full Sample

| Bin | Completers | Non Completers |
|---|---|---|
| Green | 62 | 4 |
| Yellow | 206 | 28 |
| Red | 39 | 13 |

Chi-squared X = 9.7761 P = 0.007536

TABLE 46
PD Model 1 Evaluation, Full Sample

| | Risk | | | |
|---|---|---|---|---|
| | Non | | | |
| Test Result | Completer | N | Completer | N |
| Positive (Red) | True Positive | 13 | False Positive | 39 |
| Negative | False Negative | 32 | True Negative | 268 |
| (Green or | | | | |
| Yellow) | | | | |
| | | | Disease | |
| | Evaluation | % | Prevalence (%) | |
| | Sensitivity | 28.89 | 12.78 | |
| | Specificity | 87.30 | | |

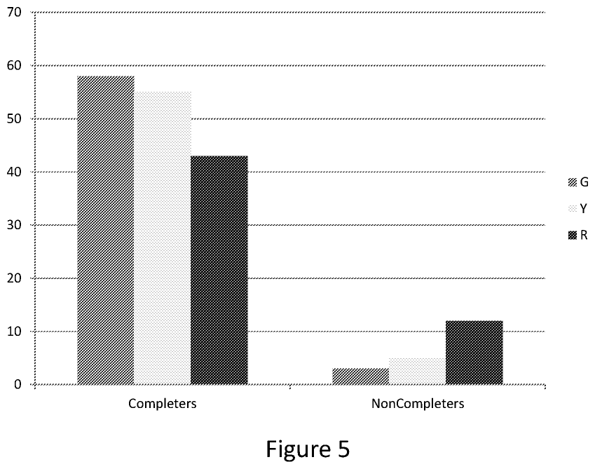

The results for the PD Model 2 are shown in FIG. 5 and Tables 47-53. FIG. 5 and Tables 47-49 show the results for PD Model 2 for Sample 1. Tables 50-51 show the results for PD Model 2 for Sample 2. Tables 52-53 show the results for PD Model 2 for the Full Sample.

TABLE 47
Univariate and Multivariate Analysis of PD 2 genes, Sample 1

| Gene Risk | Test | Significance | Correlation |
|---|---|---|---|
| ADRA2A | (completers, | P = 0.284 | r = −0.08 |
| | risk2ADRA2A) | | |
| COMT | (completers, | P = 0.03179 | r = −0.16 |
| | risk2COMT) | | |
| OPRM1 | (completers, | P = 0.8788 | r = −0.01 |
| | risk2OPRM1) | | |
| HTR2A | (completers, risk2 | P = 0.03552 | r = −0.16 |
| | HTR2A) | | |
| SLC6A4 | (completers, | P = 0.07238 | r = −0.14 |
| | risk2SLC6A4) | | |
| Risk2Sum | (completers, | P = 0.00247 | r = −0.23 |
| | Risk2Sum) | | |

TABLE 48
Binning Results for PD Model 2, Sample 1

| Bin | Risk Score | Completers | Non Completers |
|---|---|---|---|
| Green | −3.3 to −0.2 | 58 | 3 |
| Yellow | 0.2 to 1 | 55 | 5 |
| Red | 1.3 to 3.7 | 43 | 12 |

TABLE 49
PD Model 2 Evaluation, Sample 1

| | Risk | | | |
|---|---|---|---|---|
| | Non | | | |
| Test Result | Completer | N | Completer | N |
| Positive (Red) | True Positive | 12 | False Positive | 43 |
| Negative | False Negative | 8 | True Negative | 113 |
| (Green or | | | | |
| Yellow) | | | | |
| | | | Disease | |
| | Evaluation | % | Prevalence (%) | |
| | Sensitivity | 60.00 | 11.36 | |
| | Specificity | 72.44 | | |

TABLE 50
Univariate and Multivariate Analysis of PD 2 genes, Sample 2

| Gene Risk | Test | Significance | Correlation |
|---|---|---|---|
| ADRA2A | (completers, | P = 0.4277 | r = −0.06 |
| | risk2ADRA2A) | | |
| COMT | (completers, | P = 0.1324 | r = −0.11 |
| | risk2COMT) | | |
| OPRM1 | (completers, | P = 0.1659 | r = −0.10 |
| | risk2OPRM1) | | |
| HTR2A | (completers, risk2 | P = 0.06037 | r = −0.14 |
| | HTR2A) | | |
| SLC6A4 | (completers, | P = 0.06376 | r = −0.14 |
| | risk2SLC6A4) | | |
| Risk2Sum | (completers, | P = 0.00109 | r = −0.24 |
| | Risk2Sum) | | |

TABLE 51
Binning Results for PD Model 2, Sample 2

| Bin | Risk Score | Completers | Non Completers |
|---|---|---|---|
| Green | −3.2 to −1 | 70 | 2 |
| Yellow | −0.9 to 0.3 | 57 | 6 |
| Red | 0.5 to 2.7 | 32 | 9 |

TABLE 52
Univariate and Multivariate Analysis of PD 2 genes, Full Sample

| Gene Risk | Test | Significance | Correlation |
|---|---|---|---|
| ADRA2A | (completers, | P = 0.5983 | r = −0.03 |
| | risk2ADRA2A) | | |
| COMT | (completers, | P = 0.04664 | r = −0.11 |
| | risk2COMT) | | |
| OPRM1 | (completers, | P = 0.662 | r = 0.02 |
| | risk2OPRM1) | | |
| HTR2A | (completers, risk2 | P = 0.3124 | r = −0.05 |
| | HTR2A) | | |
| SLC6A4 | (completers, | P = 0.01917 | r = −0.12 |
| | risk2SLC6A4) | | |
| Risk2Sum | (completers, | P = 0.0277 | r = −0.12 |
| | Risk2Sum) | | |

TABLE 53
Binning Results for PD Model 2, Full Sample

| Bin | Risk Score | Completers | Non Completers |
|---|---|---|---|
| Green | −4.2 to −0.2 | 101 | 7 |
| Yellow | 0.1 to 1 | 120 | 18 |
| Red | 1.3+ | 86 | 20 |

Chi-squared X = 7.3748 P = 0.02504

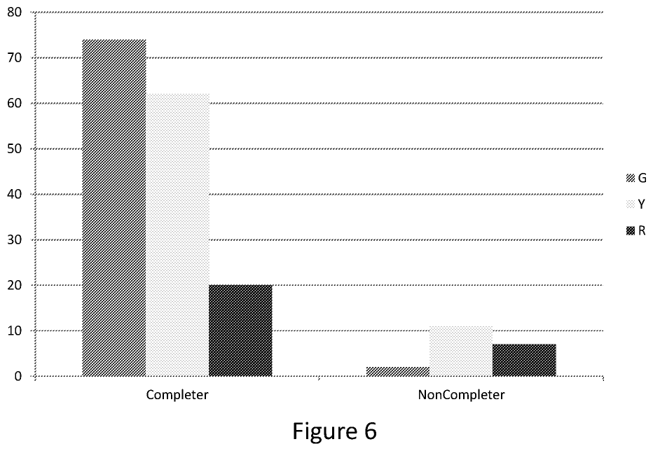

The results for the PD Model 3 are shown in FIG. 6 and Tables 54-60. FIG. 6 and Tables 54-56 show the results for PD Model 3 for Sample 1. Tables 57-58 show the results for PD Model 3 for Sample 2. Tables 59-60 show the results for PD Model 3 for the Full Sample.

TABLE 54
Univariate and Multivariate Analysis of PD 3 genes, Sample 1

| Gene Risk | Test | Significance | Correlation |
|---|---|---|---|
| ADRA2A | (completers, | P = 0.2649 | r = −0.08 |
| | risk3ADRA2A) | | |
| COMT | (completers, | P = 0.03179 | r = −0.16 |
| | risk3COMT) | | |
| OPRM1 | (completers, | P = 0.8788 | r = −0.01 |
| | risk3OPRM1) | | |
| HTR2A | (completers, risk3 | P = 0.03552 | r = −0.16 |
| | HTR2A) | | |
| SLC6A4 | (completers, | P = 0.07079 | r = −0.14 |
| | risk3SLC6A4) | | |
| Risk3Sum | (completers, | P = 0.0003594 | r = −0.27 |
| | Risk3Sum) | | |

TABLE 55
Binning Results for PD Model 3, Sample 1

| Bin | Risk Score | Completers | Non Completers |
|---|---|---|---|
| Green | −1.8 to −0.3 | 74 | 2 |
| Yellow | −0.2 to 0.9 | 62 | 11 |
| Red | 1 to 2.1 | 20 | 7 |

TABLE 56
PD Model 3 Evaluation, Sample 1

| | Risk | | | |
|---|---|---|---|---|
| | Non | | | |
| Test Result | Completer | N | Completer | N |
| Positive (Red) | True Positive | 7 | False Positive | 20 |
| Negative | False Negative | 13 | True Negative | 136 |
| (Green or | | | | |
| Yellow) | | | | |
| | | | Disease | |
| | Evaluation | % | Prevalence (%) | 1 |
| | Sensitivity | 35.00 | 11.36 | |
| | Specificity | 87.18 | | |

TABLE 57
Univariate and Multivariate Analysis of PD 3 senes. Sample 2

| Gene Risk | Test | Significance | Correlation |
|---|---|---|---|
| ADRA2A | (completers, | P = 0.4106 | r = −0.06 |
| | risk3ADRA2A) | | |
| COMT | (completers, | P = 0.1321 | r = −0.11 |
| | risk3COMT) | | |
| OPRM1 | (completers, | P = 0.1645 | r = −0.11 |
| | risk3OPRM1) | | |
| HTR2A | (completers, risk3 | P = 0.05796 | r = −0.14 |
| | HTR2A) | | |
| SLC6A4 | (completers, | P = 0.06284 | r = −0.14 |
| | risk3SLC6A4) | | |
| Risk3Sum | (completers, | P = 0.0004916 | r = −0.26 |
| | Risk3Sum) | | |

TABLE 58
Binning Results for PD Model 3, Sample 2

| Bin | Risk Score | Completers | Non Completers |
|---|---|---|---|
| Green | −2.1 to −0.7 | 77 | 2 |
| Yellow | −0.6 to 0.1 | 53 | 6 |
| Red | 0.2 to 2 | 29 | 9 |

TABLE 59
Univariate and Multivariate Analysis of PD 3 genes, Full Sample

| Gene Risk | Test | Significance | Correlation |
|---|---|---|---|
| ADRA2A | (completers, | P = 0.6314 | r = −0.03 |
| | risk3ADRA2A) | | |
| COMT | (completers, | P = 0.04664 | r = −0.11 |
| | risk3COMT) | | |
| OPRM1 | (completers, | P = 0.662 | r = 0.02 |
| | risk3OPRM1) | | |
| HTR2A | (completers, risk3 | P = 0.306 | r = −0.05 |
| | HTR2A) | | |
| SLC6A4 | (completers, | P = 0.02 | r = -0.12 |
| | risk3SLC6A4) | | |
| Risk3Sum | (completers, | P = 0.00317 | r = −0.16 |
| | Risk3Sum) | | |

TABLE 60
Binning Results for PD Model 3, Full Sample

| Bin | Risk Score | Completers | Non Completers |
|---|---|---|---|
| Green | −2.3 to −0.3 | 137 | 9 |
| Yellow | −0.2 to 0.9 | 131 | 26 |
| Red | 1+ | 39 | 10 |

Chi-squared X = 10.301 P = 0.005797

For PK/PD Model 1, final score was the sum of the PD 1 score and the PK score, where PK green=1, PK Yellow=2 and PK red=3. Bins were assigned according to Table 61.

TABLE 61
Score Conversions PK/PD Model 1

| Score | Bin |
|---|---|
| 0-2 | Green |
| 3 | Yellow |
| 4 | Red |

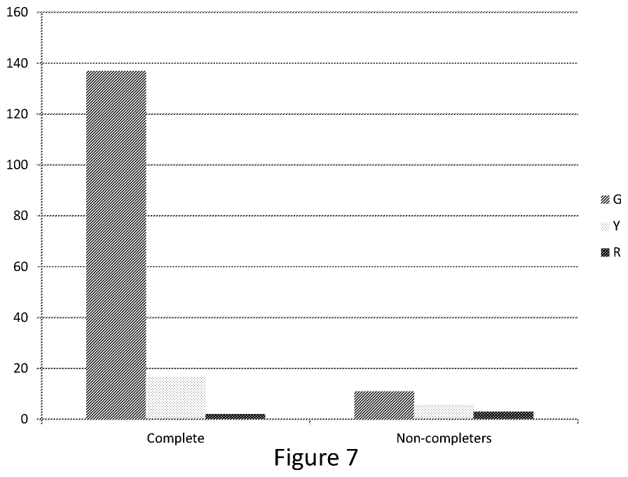

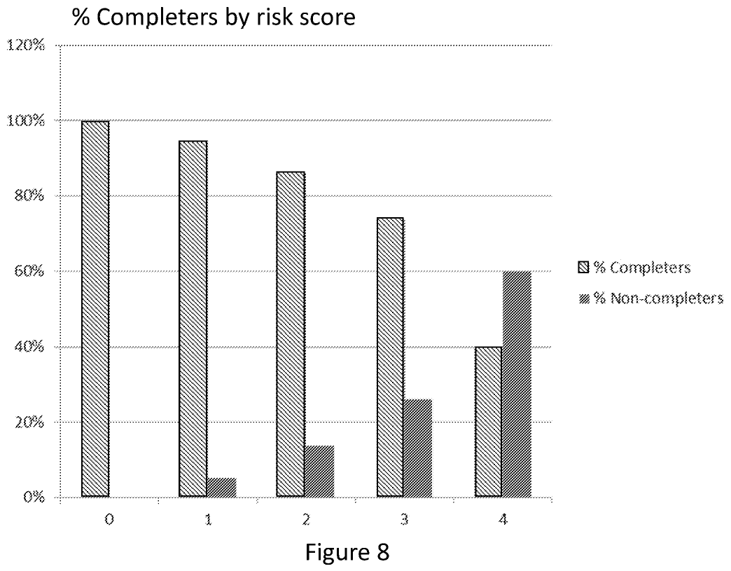

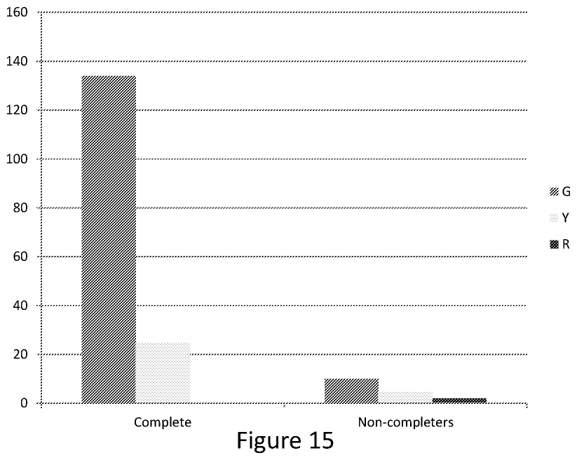

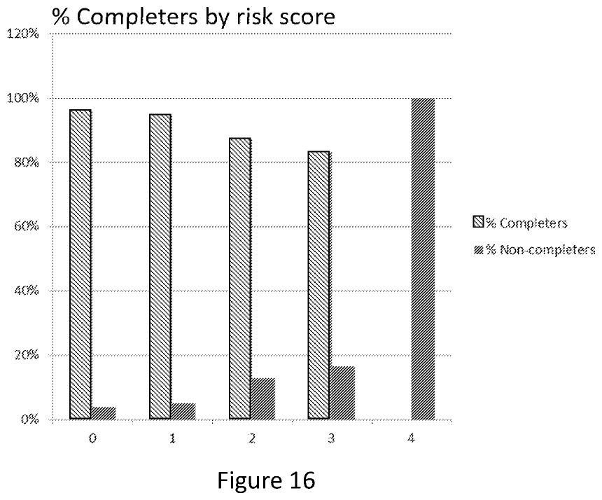

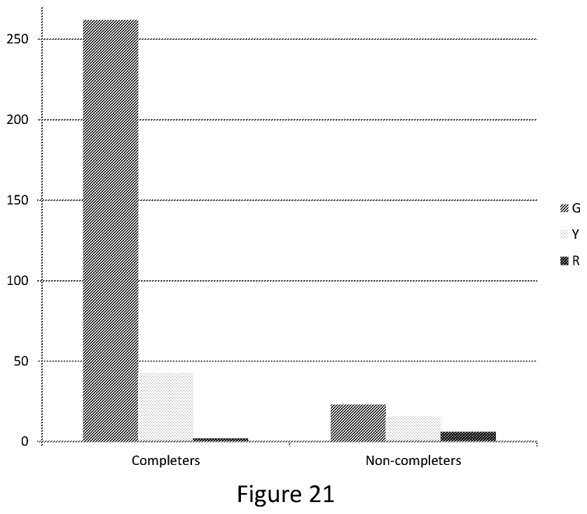

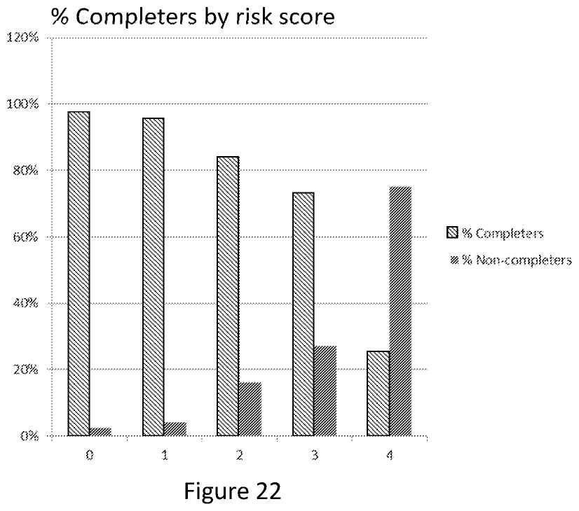

The results for PK/PD Model 1 are shown in FIGS. 7, 8, 15, 16, 21 and 22 and Tables 62-67. FIGS. 7-8 and Tables 62-63 show the results for PK/PD Model 1 for Sample 1. FIGS. 15-16 and Tables 64-65 show the results for PK/PD Model 1 for Sample 2. FIGS. 21-22 and Tables 66-67 show the results for PK/PD Model 1 for the Full Sample.

TABLE 62
Binning Results for PK/PD Model 1, Sample 1

| Bin | Completers | Non Completers |
|---|---|---|
| Green | 137 | 11 |
| Yellow | 17 | 6 |
| Red | 2 | 3 |

TABLE 63
PK/PD Model 1 Evaluation, Sample 1

| | Risk | | | |
|---|---|---|---|---|
| | Non | | | |
| Test Result | Completer | N | Completer | N |
| Positive (Red) | True Positive | 3 | False Positive | 2 |
| Negative | False Negative | 17 | True Negative | 154 |
| (Green or | | | | |
| Yellow) | | | | |
| | | | Disease | |
| | Evaluation | % | Prevalence (%) | |
| | Sensitivity | 15.00 | 11.36 | |
| | Specificity | 98.72 | | |

TABLE 64
Binning Results for PK/PD Model 1, Sample 2

| Bin | Completers | Non Completers |
|---|---|---|
| Green | 134 | 10 |
| Yellow | 25 | 5 |
| Red | 0 | 2 |

TABLE 65
PK/PD Model 1 Evaluation, Sample 2

| | Risk | | | |
|---|---|---|---|---|
| | Non | | | |
| Test Result | Completer | N | Completer | N |
| Positive (Red) | True Positive | 2 | False Positive | 0 |
| Negative | False Negative | 15 | True Negative | 159 |
| (Green or | | | | |
| Yellow) | | | | |
| | | | Disease | |
| | Evaluation | % | Prevalence (%) | |
| | Sensitivity | 11.76 | 9.66 | |
| | Specificity | 100.00 | | |

TABLE 66
Binning Results for PK/PD Model 1, Full Sample

| Bin | Completers | Non Completers |
|---|---|---|
| Green | 262 | 23 |
| Yellow | 43 | 16 |
| Red | 2 | 6 |

Fisher's Exact Test P = 7.94 × 10−8

TABLE 67
PK/PD Model 1 Evaluation, Full Sample

| | Risk | | | |
|---|---|---|---|---|
| | Non | | | |
| Test Result | Completer | N | Completer | N |
| Positive (Red) | True Positive | 6 | False Positive | 2 |
| Negative | False Negative | 39 | True Negative | 305 |
| (Green or | | | | |
| Yellow) | | | | |
| | | | Disease | |
| | Evaluation | % | Prevalence (%) | |
| | Sensitivity | 13.33 | 12.78 | |

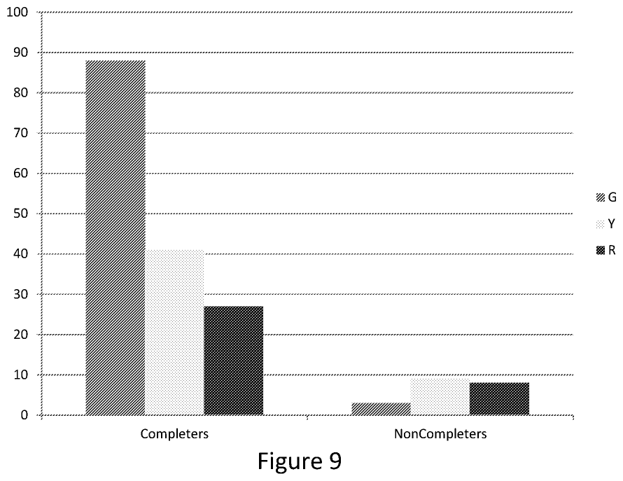

The results for PK/PD Model 2 are shown in FIG. 9 and Tables 68-73. FIG. 9 and Tables 68-69 show the results for PK/PD Model 2 for Sample 1. Table 70 shows the results for PK/PD Model 2 for Sample 2. Table 71 shows the results for PK/PD Model 2 for the Full Sample.

TABLE 68
Binning Results for PK/PD Model 2, Sample 1

| Bin | Risk Score | Completers | Non Completers |
|---|---|---|---|
| Green | −2.3 to 2 | 88 | 3 |
| Yellow | 2.2 to 3.2 | 41 | 9 |
| Red | 3.3 to 6.7 | 27 | 8 |

TABLE 69
PK/PD Model 2 Evaluation. Sample 1

| | Risk | | | |
|---|---|---|---|---|
| | Non | | | |
| Test Result | Completer | N | Completer | N |
| Positive (Red) | True Positive | 8 | False Positive | 27 |
| Negative | False Negative | 12 | True Negative | 129 |
| (Green or | | | | |
| Yellow) | | | | |
| | | | Disease | |
| | Evaluation | % | Prevalence (%) | |
| | Sensitivity | 40.00 | 11.36 | |
| | Specificity | 82.69 | | |

TABLE 70
Binning Results for PK/PD Model 2, Sample 2

| Bin | Risk Score | Completers | Non Completers |
|---|---|---|---|
| Green | −2.2 to 1 | 92 | 2 |
| Yellow | 1.1 to 2.5 | 48 | 6 |
| Red | 2.7 to 4.7 | 19 | 9 |

TABLE 71
Binning Results for PK/PD Model 2, Full Sample

| Bin | Risk Score | Completers | Non Completers |
|---|---|---|---|
| Green | −2.2 to 1 | 167 | 8 |
| Yellow | 1.1 to 2.5 | 80 | 17 |
| Red | 2.7 to 4.7 | 60 | 20 |

Chi-squared X = 23.249 P = 8.942 × 10−6

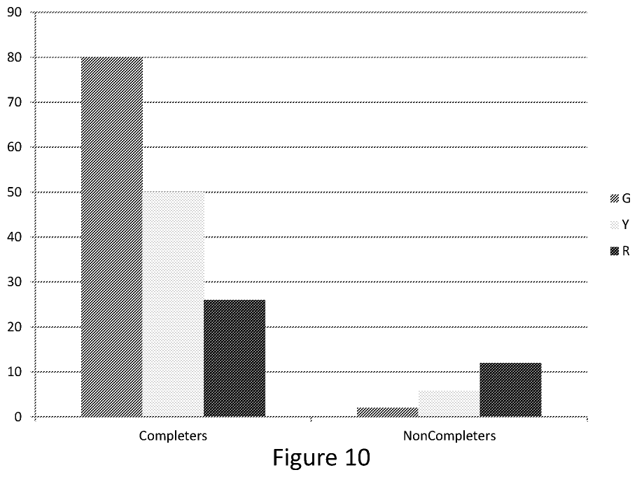

The results for PK/PD Model 3 are shown in FIG. 10 and Tables 72-75. FIG. 10 and Tables 72-73 show the results for PK/PD Model 3 for Sample 1. Table 74 shows the results for PK/PD Model 3 for Sample 2. Table 75 shows the results for PK/PD Model 3 for the Full Sample.

TABLE 72
Binning Results for PK/PD Model 3, Sample 1

| Bin | Risk Score | Completers | Non Completers |
|---|---|---|---|
| Green | −0.8 to 1.3 | 80 | 2 |
| Yellow | 1.4 to 2.3 | 50 | 6 |
| Red | 2.4 to 5.1 | 26 | 12 |

TABLE 73
PK/PD Model 3 Evaluation, Sample 1

| | Risk | | | |
|---|---|---|---|---|
| | Non | | | |
| Test Result | Completer | N | Completer | N |
| Positive (Red) | True Positive | 12 | False Positive | 26 |
| Negative | False Negative | 8 | True Negative | 130 |
| (Green or | | | | |
| Yellow) | | | | |
| | | | Disease | |
| | Evaluation | % | Prevalence (%) | |
| | Sensitivity | 60.00 | 11.36 | |

TABLE 74
Binning Results for PK/PD Model 3, Sample 2

| Bin | Risk Score | Completers | Non Completers |
|---|---|---|---|
| Green | −1.1 to 0.8 | 77 | 2 |
| Yellow | 0.9 to 2.1 | 58 | 5 |
| Red | 2.2 to 4.3 | 24 | 10 |

TABLE 75
Binning Results for PK/PD Model 3, Full Sample

| Bin | Risk Score | Completers | Non Completers |
|---|---|---|---|
| Green | −1.3 to 1.3 | 150 | 7 |
| Yellow | 1.4 to 2.3 | 93 | 10 |
| Red | 2.4+ | 64 | 28 |

Chi-squared X = 36.34 P = 1.285 × 10−8

### DISCUSSION

A summary of the results of this study are provided in Table 76. Because the binning cutoffs in PD Models 2 and 3 depend on the sample, the cutoffs determined for Sample 2 and Sample 3 are both represented in the Table, along with their corresponding PK/PD models (e.g. PD2_1 and PD2_2 are the two variants of PD Model 2).

TABLE 76
Model Summary

| | | | | Sensitivity | | | |
|---|---|---|---|---|---|---|---|
| Sample | Model_Sample | p_value | R² | (%) | Specificity (%) | PPV (%) | NPV (%) |
| Full | Current | 0.1306 | 0.006 | 6.67 | 90.88 | 9.68 | 86.92 |
| Full | PKPD1 | 6.31 × 10−11 | 0.121 | 13.33 | 99.35 | 75 | 88.66 |
| Full | PD3_1 | 0.0056 | 0.024 | 22.22 | 87.3 | 20.41 | 88.45 |
| Full | PD1 | 0.007 | 0.022 | 28.89 | 87.3 | 25 | 89.33 |
| Full | PKPD2_2 | 1.53 × 10−6 | 0.069 | 40 | 86.32 | 30 | 90.75 |
| Full | PD2_1 | 0.0249 | 0.015 | 44.44 | 71.99 | 18.87 | 89.84 |
| Full | PD2_2 | 0.018 | 0.018 | 44.44 | 75.57 | 21.05 | 90.27 |
| Full | PD3_2 | 0.0105 | 0.020 | 44.44 | 76.22 | 21.51 | 90.35 |
| Full | PKPD2_1 | 6.63 × 10−6 | 0.061 | 44.44 | 80.46 | 25 | 90.81 |
| Full | PK1 | 3.67 × 10−11 | 0.124 | 48.89 | 89.25 | 40 | 92.26 |
| Full | PKPD3_2 | 1.92 × 10−7 | 0.080 | 53.33 | 82.08 | 30.38 | 92.31 |
| Full | PKPD3_1 | 5.52 × 10−9 | 0.100 | 62.22 | 79.15 | 30.43 | 93.46 |

Given the frequency of non-completers in the tested population, PPV and NPV (positive and negative predictive value) are also calculated. However, a person skilled in the art would recognize that in a different testing population with a different frequency of non-completers, these PPV and NPV values would change. Thus, a skilled person may select a model incorporating the genes and panels disclosed in this specification based on the intended testing population. Additionally, a skilled person would also consider the intended use of the test. If the use is focused more on identifying completers in a population, a skilled person might select a model with a higher sensitivity. On the other hand, if the use is more focused on screening out non-responders, the skilled person might select a model with higher specificity. Where both the foregoing goals are important, a more balanced test, like PKPD1 might be appropriate. The present invention has been described with regard to one or more embodiments. However, it will be apparent to persons skilled in the art that a number of variations and modifications can be made without departing from the scope of the invention as defined in the claims.

What is claimed is:

1-60. (canceled)

61. A method of treating a patient with opioid addiction, the method comprising:
(a) determining the patient's genetic phenotype for one or more pharmacodynamic genes, and optionally determining the subject's genetic phenotype for one or more cytochrome P450 (CYP) genes, by obtaining or having obtained a biological sample from the patient, and performing or having performed a genotyping assay on the sample;
(b) generating a composite genetic risk score for the patient;
(c) determining if the patient has a high, intermediate or low risk of non-completion of opioid agonist replacement therapy based on the subject's composite genetic risk score, and
(d) if the patient has a high or intermediate risk for non-completion of opioid agonist replacement therapy, then administering an initial dose of buprenorphine that is higher than a starting dose determined according to clinical guidelines or administering methadone to said patient, and if the patient does not have a high or intermediate risk for non-completion of opioid agonist replacement therapy, then administering an initial dose of buprenorphine at starting dose determined according to clinical guidelines, wherein a risk of dropout for a patient with high or intermediate risk of non-completion is lower following the administration of a higher initial dose of buprenorphine or the administration of methadone, than if buprenorphine were administered at a starting dose according to clinical guidelines.

62. The method of claim 61, wherein if the patient has a high risk for non-completion of opioid agonist replacement therapy, then administering methadone to said patient, if the patient has an intermediate risk for non-completion of opioid agonist replacement therapy, then administering an initial dose of buprenorphine that is higher than a starting dose determined according to clinical guidelines or administering methadone to said patient, and if the patient does not have a high or intermediate risk for non-completion of opioid agonist replacement therapy, then administering an initial dose of buprenorphine at starting dose determined according to clinical guidelines, wherein a risk of dropout for a patient with high or intermediate risk of non-completion is lower following the administration of a higher initial dose of buprenorphine or the administration of methadone, than if buprenorphine were administered at a starting dose according to clinical guidelines

63. The method of claim 61, wherein said one or more pharmacodynamic genes are selected from the group consisting of ADRA2A, COMT, HTR2A, OPRM1, and SLC6A4.

64. The method of claim 61, wherein said one or more cytochrome P450 genes are selected from the group consisting of CYP1A, CYP2B6, CYP2C9, CYP2C19, and CYP2D6.

65. The method of claim 61, wherein the composite genetic risk score for the subject comprises the sum of the risks associated with each genetic phenotype.

66. The method of claim 61, wherein the genetic phenotype of each of the one or more CYP genes is a combination phenotype based upon the number of functional alleles at the genetic locus.

67. The method of claim 61, wherein the subject's genetic phenotype is determined for a panel of two genes selected from the group of panels consisting of
COMT, CYP3A4;
COMT, HTR2A;
COMT, SLC6A4;
HTR2A, CYP3A4;
SLC6A4, HTR2A; and
SLC6A4, CYP3A4.

68. The method of claim 61, wherein the subject's genetic phenotype is determined for a panel of genes selected from the group of panels consisting of
COMT, HTR2A, CYP3A4;
COMT, SLC6A4, CYP3A4;
COMT, SLC6A4, HTR2A; and
SLC6A4, HTR2A, CYP3A4.

69. The method of claim 61, wherein the subject's genetic phenotype is determined for a panel of genes selected from the group of panels consisting of
COMT, SLC6A4, HTR2A, CYP3A4;
COMT, SLC6A4, HTR2A, CYP3A4, CYP2B6;
COMT, SLC6A4, HTR2A, CYP3A4, CYP2C19;
COMT, SLC6A4, HTR2A, CYP3A4, CYP1A2;
COMT, SLC6A4, HTR2A, CYP3A4, CYP2C9; and
COMT, SLC6A4, HTR2A, CYP3A4, CYP2D6.

70. The method of claim 61, wherein the subject's genetic phenotype is determined for a panel of genes selected from the group of panels consisting of
COMT, SLC6A4, HTR2A, CYP3A4, CYP2B6, CYP2C19;
COMT, SLC6A4, HTR2A, CYP3A4, CYP2B6, CYP1A2;
COMT, SLC6A4, HTR2A, CYP3A4, CYP2B6, CYP2C9;
COMT, SLC6A4, HTR2A, CYP3A4, CYP2B6, CYP2D6;
COMT, SLC6A4, HTR2A, CYP3A4, CYP2C19, CYP1A2;
COMT, SLC6A4, HTR2A, CYP3A4, CYP2C19, CYP2C9; and
COMT, SLC6A4, HTR2A, CYP3A4, CYP2C19, CYP2D6.

71. The method of claim 61, wherein the subject's genetic phenotype is determined for a panel of genes selected from the group consisting of
COMT, SLC6A4, HTR2A, CYP3A4, CYP2B6, CYP2C9, CYP2D6;
COMT, SLC6A4, HTR2A, CYP3A4, CYP2B6, CYP2C9, CYP2D6, CYP2C19, and CYP1A2.

72. The method of claim 61, wherein the genetic phenotype is determined by assaying for one or more of the following genetic variants in the one or more pharmacodynamic genes: ADRA2A (r51800544), COMT (r54680), HTR2A (rs6311), OPRM1 (r51799971), SLC6A4 (5-HTTLPR).

73. The method of claim 61, wherein the genetic phenotype is assigned based on the genotype at each genetic variant.

74. The method of claim 61, wherein determining the subject's genetic phenotype for one or more cytochrome P450 (CYP) genes comprises
combining combinatorial pharmacogenetics with a physiological algorithm for predicting medication exposure.

75. The method of claim 61, wherein determining the subject's genetic phenotype for one or more pharmacodynamic genes comprises
assigning a numerical score to each genotype associated with a pharmacodynamic gene based on the difference between the completion percentage for that genotype and the mean completion percentage in a reference population.

76. The method of claim 61, further comprising identifying the subject as having a high, intermediate, or low risk of non-completion of opioid agonist replacement therapy based on the subject's composite genetic risk score as follows
if the composite genetic risk score has a p-value of less than or equal to 0.05 and an odds ratio greater than or equal to 1.5, the subject is at high risk;
if the composite genetic risk score has a p-value of less than or equal to 0.05 and an odds ratio between 1 and 1.5, the subject is at intermediate risk; and
if none of the above conditions are met, the subject is at low risk.
